(19) United States
(12) Patent Application Publication
(10) Pub. No.: US 2007/0249667 A1
(43) Pub. Date: Oct. 25, 2007

(54) Use of Substituted Quinoline Derivatives for the Treatment of Drug Resistant Mycobacterial Diseases

(75) Inventors: Koenraad Jozef Lodewijk Marcel Andries, Beerse (BE); Jozef Frans Elisabetha Van Gestel, Vosselaar (BE)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: JANSSEN PHARMACEUTICA N.V., Beerse (BE)

(21) Appl. No.: 11/569,681

(22) PCT Filed: May 24, 2005
(86) PCT No.: PCT/EP05/52371

§ 371 Date: Nov. 28, 2006

(30) Foreign Application Priority Data
May 28, 2004 (EP) 04102402.7

(51) Int. Cl. A61K 31/44; A61P 43/00

(52) U.S. Cl. 514/311

(57) ABSTRACT

The present invention relates to the use of a substituted quinoline derivative for the preparation of a medicament for the treatment of an infection with a drug resistant Mycobacterium strain wherein the substituted quinoline derivative is a compound according to Formula (Ia) or Formula (Ib) 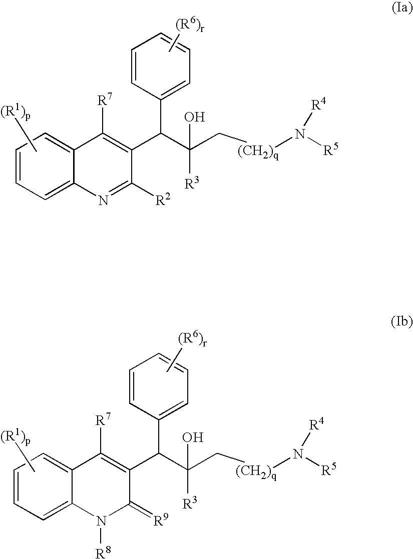 the pharmaceutically acceptable acid or base addition salts thereof, the stereochemically isomeric forms thereof, the tautomeric forms thereof and the N-oxide forms thereof. Also claimed is a composition comprising a pharmaceutically acceptable carrier and, as active ingredient, a therapeutically effective amount of the above compounds and one or more other antimycobacterial agents.

The present invention relates to the use of substituted quinoline derivatives for inhibiting the growth of drug resistant Mycobacterium strains including growth inhibition of multi drug resistant Mycobacterium strains. The substituted quinoline derivatives can thus be used for the treatment or the prevention of Mycobacterial diseases caused by drug resistant, particularly multi drug resistant Mycobacteria. More in particular the present quinoline derivatives can be used for the treatment or the prevention of Mycobacterial diseases caused by drug resistant including multi drug resistant Mycobacterium tuberculosis. The present invention also relates to a combination of (a) a substituted quinoline derivative according to the present invention and (b) one or more other antimycobacterial agents.

### BACKGROUND OF THE INVENTION

Mycobacterium tuberculosis is the causative agent of tuberculosis (TB), a serious and potentially fatal infection with a world-wide distribution. Estimates from the World Health Organization indicate that more than 8 million people contract TB each year, and 2 million people die from tuberculosis yearly. In the last decade, TB cases have grown 20% worldwide with the highest burden in the most impoverished communities. If these trends continue, TB incidence will increase by 41% in the next twenty years. Fifty years since the introduction of an effective chemotherapy, TB remains after AIDS, the leading infectious cause of adult mortality in the world. Complicating the TB epidemic is the rising tide of multi-drug-resistant strains, and the deadly symbiosis with HIV. People who are HIV-positive and infected with TB are 30 times more likely to develop active TB than people who are HIV-negative and TB is responsible for the death of one out of every three people with HIV/AIDS worldwide

Existing approaches to treatment of tuberculosis all involve the combination of multiple agents. For example, the regimen recommended by the U.S. Public Health Service is a combination of isoniazid, rifampicin and pyrazinamide for two months, followed by isoniazid and rifampicin alone for a further four months. These drugs are continued for a further seven months in patients infected with HIV. For patients infected with multi-drug resistant strains of M. tuberculosis, agents such as ethambutol, streptomycin, kanamycin, amikacin, capreomycin, ethionamide, cycloserine, ciprofoxacin and ofloxacin are added to the combination therapies. There exists no single agent that is effective in the clinical treatment of tuberculosis, nor any combination of agents that offers the possibility of therapy of less than six months' duration.

There is a high medical need for new drugs that improve current treatment by enabling regimens that facilitate patient and provider compliance. Shorter regimens and those that require less supervision are the best way to achieve this. Most of the benefit from treatment comes in the first 2 months, during the intensive, or bactericidal, phase when four drugs are given together; the bacterial burden is greatly reduced, and patients become noninfectious. The 4- to 6-month continuation, or sterilizing, phase is required to eliminate persisting bacilli and to minimize the risk of relapse. A potent sterilizing drug that shortens treatment to 2 months or less would be extremely beneficial. Drugs that facilitate compliance by requiring less intensive supervision also are needed. Obviously, a compound that reduces both the total length of treatment and the frequency of drug administration would provide the greatest benefit.

Complicating the TB epidemic is the increasing incidence of multi drug-resistant strains or MDR-TB. Up to four percent of all cases worldwide are considered MDR-TB—those resistant to the most effective drugs of the four-drug standard, isoniazid and rifampin. MDR-TB is lethal when untreated and can not be adequately treated through the standard therapy, so treatment requires up to 2 years of “second-line” drugs. These drugs are often toxic, expensive and marginally effective. In the absence of an effective therapy, infectious MDR-TB patients continue to spread the disease, producing new infections with MDR-TB strains. There is a high medical need for drugs which demonstrate activity against resistant and/or MDR strains.

The term “drug resistant” as used hereinbefore or hereinafter is a term well understood by the person skilled in microbiology. A drug resistant Mycobacterium is a Mycobacterium which is no longer susceptible to at least one previously effective drug; which has developed the ability to withstand antibiotic attack by at least one previously effective drug. A drug resistant strain may relay that ability to withstand to its progeny. Said resistance may be due to random genetic mutations in the bacterial cell that alters its sensitivity to a single drug or to different drugs.

MDR tuberculosis is a specific form of drug resistant tuberculosis due to a bacterium resistant to at least isoniazid and rifampicin (with or without resistance to other drugs), which are at present the two most powerful anti-TB drugs. Thus, whenever used hereinbefore or hereinafter “drug resistant” includes multi drug resistant.

Unexpectedly, it has now been found that the substituted quinoline derivatives of the present invention are very useful for inhibiting growth of drug resistant, in particular multi drug resistant, Mycobacteria and therefore useful for the treatment of diseases caused by drug resistant, in particular multi drug resistant, Mycobacteria, particularly those diseases caused by drug resistant, in particular multi drug resistant, pathogenic Mycobacterium (M.) tuberculosis, M. bovis, M. avium, M. fortuitum, M. leprae and M. marinum, more particularly Mycobacterium tuberculosis.

The substituted quinoline derivatives relating to the present invention were already disclosed in WO 2004/011436. Said document discloses the antimycobacterial property of the substituted quinoline derivatives against sensitive, susceptible Mycobacterium strains but is silent on their activity against drug resistant, in particular multi drug resistant, Mycobacteria.

Thus, the present invention relates to the use of a substituted quinoline derivative for the preparation of a medicament for the treatment of a warm-blooded mammal infected with a drug resistant Mycobacterium strain wherein the substituted quinoline derivative is a compound according to Formula (Ia) or Formula (Ib) 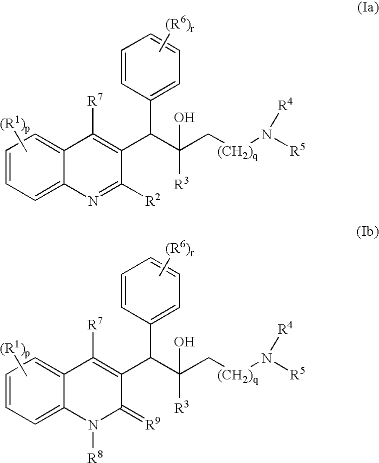
a pharmaceutically acceptable acid or base addition salt thereof, a stereochemically isomeric form thereof, a tautomeric form thereof or a N-oxide form thereof, wherein:
R¹ is hydrogen, halo, haloalkyl, cyano, hydroxy, Ar, Het, alkyl, alkyloxy, alkylthio, alkyloxyalkyl, alkylthioalkyl, Ar-alkyl or di(Ar)alkyl;
p is an integer equal to 1, 2, 3 or 4;
R² is hydrogen, hydroxy, mercapto, alkyloxy, alkyloxyalkyloxy, alkylthio, mono or di(alkyl)amino or a radical of formula 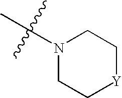
   wherein Y is CH₂, O, S, NH or N-alkyl;
R³ is alkyl, Ar, Ar-alkyl Het or Het-alkyl;
q is an integer equal to zero, 1, 2, 3 or 4;
R⁴ and R⁵ each independently are hydrogen, alkyl or benzyl; or
R⁴ and R⁵ together and including the N to which they are attached may form a radical selected from the group of pyrrolidinyl, 2H-pyrrolyl 2-pyrrolinyl, 3-pyrrolinyl pyrrolyl imidazolidinyl, pyrazolidinyl, 2-imidazolinyl, 2-pyrazolinyl, imidazolyl, pyrazolyl, triazolyl, piperidinyl, pyridinyl, piperazinyl, imidazolidinyl, pyridazinyl, pyrimidinyl, pyrazinyl, triazinyl, morpholinyl and thiomorpholinyl, optionally substituted with alkyl, halo, haloalkyl, hydroxy, alkyloxy, amino, mono- or dialkylamino, alkylthio, alkyloxyalkyl, alkylthioalkyl and pyrimidinyl;
R⁶ is hydrogen, halo, haloalkyl, hydroxy, Ar, alkyl, alkyloxy, alkylthio, alkyloxyalkyl alkylthioalkyl Ar-alkyl or di(Ar)alkyl; or
two vicinal R⁶ radicals may be taken together to form a bivalent radical of formula —CH═CH—CH═CH—;
r is an integer equal to 1, 2, 3, 4 or 5; and
R⁷ is hydrogen, alkyl, Ar or Het;
R⁸ is hydrogen or alkyl;
R⁹ is oxo; or
R⁸ and R⁹ together form the radical ═N—CH═CH—;
alkyl is a straight or branched saturated hydrocarbon radical having from 1 to 6 carbon atoms; or is a cyclic saturated hydrocarbon radical having from 3 to 6 carbon atoms; or is a a cyclic saturated hydrocarbon radical having from 3 to 6 carbon atoms attached to a straight or branched saturated hydrocarbon radical having from 1 to 6 carbon atoms; wherein each carbon atom can be optionally substituted with halo, hydroxy, alkyloxy or oxo;
Ar is a homocycle selected from the group of phenyl, naphthyl, acenaphthyl, tetrahydronaphthyl, each optionally substituted with 1, 2 or 3 substituents, each substituent independently selected from the group of hydroxy, halo, cyano, nitro, amino, mono- or dialkylamino, alkyl, haloalkyl, alkyloxy, haloalkyloxy, carboxyl, alkyloxycarbonyl, aminocarbonyl, morpholinyl and mono- or dialkylaminocarbonyl;
Het is a monocyclic heterocycle selected from the group of N-phenoxypiperidinyl, pyrrolyl, pyrazolyl, imidazolyl, furanyl, thienyl, oxazolyl, isoxazolyl, thiazolyl, isothiazolyl, pyridinyl, pyrimidinyl, pyrazinyl and pyridazinyl; or a bicyclic heterocycle selected from the group of quinolinyl, quinoxalinyl, indolyl, benzimidazolyl, benzoxazolyl, benzisoxazolyl, benzothiazolyl, benzisothiazolyl, benzofuranyl, benzothienyl, 2,3-dihydrobenzo[1,4]dioxinyl or benzo[1,3]dioxolyl; each monocyclic and bicyclic heterocycle may optionally be substituted on a carbon atom with 1, 2 or 3 substituents selected from the group of halo, hydroxy, alkyl or alkyloxy;
halo is a substituent selected from the group of fluoro, chloro, bromo and iodo and
haloalkyl is a straight or branched saturated hydrocarbon radical having from 1 to 6 carbon atoms or a cyclic saturated hydrocarbon radical having from 3 to 6 carbon atoms, wherein one or more carbon atoms are substituted with one or more halo-atoms.

More in particular, the present invention relates to the use of a substituted quinoline derivative for the preparation of a medicament for the treatment of an infection with a drug resistant Mycobacterium strain wherein the substituted quinoline derivative is a compound according to Formula (Ia) or Formula (Ib).

The present invention also concerns a method of treating a patient suffering from, or at risk of, an infection with a drug resistant mycobacterial strain, which comprises administering to the patient a therapeutically effective amount of a compound or pharmaceutical composition according to the invention.

The compounds according to Formula (Ia) and (Ib) are interrelated in that e.g. a compound according to Formula (Ib), with R⁹ equal to oxo is the tautomeric equivalent of a compound according to Formula (Ia) with R² equal to hydroxy (keto-enol tautomerism).

In the framework of this application, alkyl is a straight or branched saturated hydrocarbon radical having from 1 to 6 carbon atoms; or is a cyclic saturated hydrocarbon radical having from 3 to 6 carbon atoms; or is a cyclic saturated hydrocarbon radical having from 3 to 6 carbon atoms attached to a straight or branched saturated hydrocarbon radical having from 1 to 6 carbon atoms; wherein each carbon atom can be optionally substituted with halo, hydroxy, alkyloxy or oxo. Preferably, alkyl is methyl, ethyl or cyclohexylmethyl.

In the framework of this application, Ar is a homocycle selected from the group of phenyl, naphthyl, acenaphthyl, tetrahydronaphthyl, each optionally substituted with 1, 2 or 3 substituents, each substituent independently selected from the group of hydroxy, halo, cyano, nitro, amino, mono- or dialkylamino, alkyl haloalkyl, alkyloxy, haloalkyloxy, carboxyl, alkyloxycarbonyl, aminocarbonyl, morpholinyl and mono- or dialkylaminocarbonyl. Preferably, Ar is naphthyl or phenyl, each optionally substituted with 1 or 2 halo substituents.

In the framework of this application, Het is a monocyclic heterocycle selected from the group of N-phenoxypiperidinyl, pyrrolyl, pyrazolyl, imidazolyl, furanyl, thienyl, oxazolyl, isoxazolyl, thiazolyl, isothiazolyl, pyridinyl, pyrimidinyl, pyrazinyl and pyridazinyl; or a bicyclic heterocycle selected from the group of quinolinyl, quinoxalinyl, indolyl, benzimidazolyl, benzoxazolyl, benzisoxazolyl, benzothiazolyl, benzisothiazolyl, benzofuranyl, benzothienyl, 2,3-dihydrobenzo[1,4]dioxinyl or benzo[1,3]dioxolyl; each monocyclic and bicyclic heterocycle may optionally be substituted on a carbon atom with 1, 2 or 3 substituents selected from the group of halo, hydroxy, alkyl or alkyloxy. Preferably, Het is thienyl.

In the framework of this application, halo is a substituent selected from the group of fluoro, chloro, bromo and iodo and haloalkyl is a straight or branched saturated hydrocarbon radical having from 1 to 6 carbon atoms or a cyclic saturated hydrocarbon radical having from 3 to 6 carbon atoms, wherein one or more carbon atoms are substituted with one or more halo-atoms. Preferably, halo is bromo, fluoro or chloro and preferably, haloalkyl is trifluoromethyl. When alkyl is substituted with more than one halo atom, each halo atom may be the same or different.

Preferably, the invention relates to the use as defined hereinabove of compounds of Formula (Ia) or (Ib) 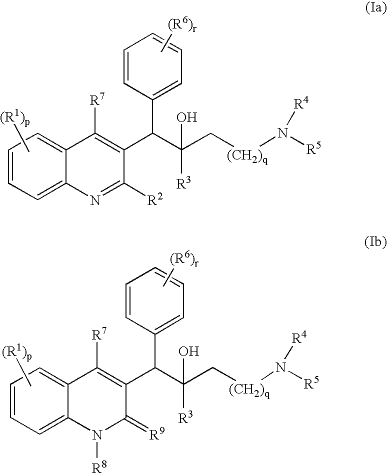
a pharmaceutically acceptable acid or base addition salt thereof, a stereochemically isomeric form thereof, a tautomeric form thereof or a N-oxide form thereof, wherein:
R¹ is hydrogen, halo, haloalkyl, cyano, hydroxy, Ar, Het, alkyl, alkyloxy, alkylthio, alkyloxyalkyl, alkylthioalkyl, Ar-alkyl or di(Ar)alkyl;
p is an integer equal to 1, 2, 3 or 4;
R² is hydrogen, hydroxy, mercapto, alkyloxy, alkyloxyalkyloxy, alkylthio, mono or di(alkyl)amino or a radical of formula 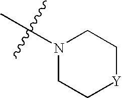
   wherein Y is CH₂, O, S, NH or N-alkyl;
R³ is alkyl, Ar, Ar-alkyl, Het or Het-alkyl;
q is an integer equal to zero, 1, 2, 3 or 4;
R⁴ and R⁵ each independently are hydrogen, alkyl or benzyl; or
R⁴ and R⁵ together and including the N to which they are attached may form a radical selected from the group of pyrrolidinyl, 2H-pyrrolyl, 2-pyrrolinyl, 3-pyrrolinyl, pyrrolyl, imidazolidinyl, pyrazolidinyl, 2-imidazolinyl, 2-pyrazolinyl, imidazolyl, pyrazolyl, triazolyl, piperidinyl, pyridinyl, piperazinyl, imidazolidinyl, pyridazinyl, pyrimidinyl, pyrazinyl, triazinyl, morpholinyl and thiomorpholinyl, optionally substituted with alkyl, halo, haloalkyl, hydroxy, alkyloxy, amino, mono- or dialkylamino, alkylthio, alkyloxyalkyl, alkylthioalkyl and pyrimidinyl;
R⁶ is hydrogen, halo, haloalkyl, hydroxy, Ar, alkyl, alkyloxy, alkylthio, alkyloxyalkyl, alkylthioalkyl, Ar-alkyl or di(Ar)alkyl; or
two vicinal R⁶ radicals may be taken together to form a bivalent radical of formula ═C—C═C═C—;
r is an integer equal to 1, 2, 3, 4 or 5; and
R⁷ is hydrogen, alkyl, Ar or Het;
R⁸ is hydrogen or alkyl;
R⁹ is oxo; or
R⁸ and R⁹ together form the radical ═N—CH═CH—;
alkyl is a straight or branched saturated hydrocarbon radical having from 1 to 6 carbon atoms; or is a cyclic saturated hydrocarbon radical having from 3 to 6 carbon atoms; or is a a cyclic saturated hydrocarbon radical having from 3 to 6 carbon atoms attached to a straight or branched saturated hydrocarbon radical having from 1 to 6 carbon atoms; wherein each carbon atom can be optionally substituted with halo, hydroxy, alkyloxy or oxo;
Ar is a homocycle selected from the group of phenyl, naphthyl, acenaphthyl, tetrahydronaphthyl, each optionally substituted with 1, 2 or 3 substituents, each substituent independently selected from the group of hydroxy, halo, cyano, nitro, amino, mono- or dialkylamino, alkyl, haloalkyl, alkyloxy, haloalkyloxy, carboxyl, alkyloxycarbonyl, aminocarbonyl, morpholinyl and mono- or dialkylaminocarbonyl;
Het is a monocyclic heterocycle selected from the group of N-phenoxypiperidinyl, pyrrolyl, pyrazolyl, imidazolyl, furanyl, thienyl, oxazolyl, isoxazolyl, thiazolyl, isothiazolyl, pyridinyl, pyrimidinyl, pyrazinyl and pyridazinyl; or a bicyclic heterocycle selected from the group of quinolinyl, quinoxalinyl, indolyl, benzimidazolyl, benzoxazolyl, benzisoxazolyl, benzothiazolyl, benzisothiazolyl, benzofuranyl, benzothienyl, 2,3-dihydrobenzo[1,4]dioxinyl or benzo[1,3]dioxolyl; each monocyclic and bicyclic heterocycle may optionally be substituted on a carbon atom with 1, 2 or 3 substituents selected from the group of halo, hydroxy, alkyl or alkyloxy;
halo is a substituent selected from the group of fluoro, chloro, bromo and iodo and
haloalkyl is a straight or branched saturated hydrocarbon radical having from 1 to 6 carbon atoms or a cyclic saturated hydrocarbon radical having from 3 to 6 carbon atoms, wherein one or more carbonatoms are substituted with one or more halo-atoms.

The invention also relates to the use as defined hereinabove of compounds of Formula (Ia) or (Ib) wherein
R¹ is hydrogen, halo, haloalkyl, cyano, hydroxy, Ar, Het, alkyl, alkyloxy, alkylthio, alkyloxyalkyl alkylthioalkyl, Ar-alkyl or di(Ar)alkyl;
p is an integer equal to 1, 2, 3 or 4;
R² is hydrogen, hydroxy, mercapto, alkyloxy, alkyloxyalkyloxy, alkylthio, mono or di(alkyl)amino or a radical of formula 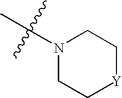
   wherein Y is CH₂, O, S, NH or N-alkyl;
R³ is alkyl, Ar, Ar-alkyl, Het or Het-alkyl;
q is an integer equal to zero, 1, 2, 3 or 4;
R⁴ and R⁵ each independently are hydrogen, alkyl or benzyl; or
R⁴ and R⁵ together and including the N to which they are attached may form a radical selected from the group of pyrrolidinyl, 2H-pyrrolyl, 2-pyrrolinyl, 3-pyrrolinyl, pyrrolyl, imidazolidinyl, pyrazolidinyl, 2-imidazolinyl, 2-pyrazolinyl, imidazolyl, pyrazolyl, triazolyl, piperidinyl, pyridinyl, piperazinyl imidazolidinyl, pyridazinyl, pyrimidinyl, pyrazinyl, triazinyl, morpholinyl and thiomorpholinyl, optionally substituted with alkyl, halo, haloalkyl, hydroxy, alkyloxy, amino, mono- or dialkylamino, alkylthio, alkyloxyalkyl, alkylthioalkyl and pyrimidinyl;
R⁶ is hydrogen, halo, haloalkyl, hydroxy, Ar, alkyl, alkyloxy, alkylthio, alkyloxyalkyl, alkylthioalkyl, Ar-alkyl or di(Ar)alkyl;
r is an integer equal to 1, 2, 3, 4 or 5; and
R⁷ is hydrogen, alkyl, Ar or Het;
R⁸ is hydrogen or alkyl;
R⁹ is oxo; or
R⁸ and R⁹ together form the radical ═N—CH═CH—;
alkyl is a straight or branched saturated hydrocarbon radical having from 1 to 6 carbon atoms; or is a cyclic saturated hydrocarbon radical having from 3 to 6 carbon atoms; or is a a cyclic saturated hydrocarbon radical having from 3 to 6 carbon atoms attached to a straight or branched saturated hydrocarbon radical having from 1 to 6 carbon atoms; wherein each carbon atom can be optionally substituted with halo, hydroxy, alkyloxy or oxo;
Ar is a homocycle selected from the group of phenyl, naphthyl, acenaphthyl, tetrahydronaphthyl, each optionally substituted with 1, 2 or 3 substituents, each substituent independently selected from the group of hydroxy, halo, cyano, nitro, amino, mono- or dialkylamino, alkyl, haloalkyl, alkyloxy, haloalkyloxy, carboxyl, alkyloxycarbonyl, aminocarbonyl, morpholinyl and mono- or dialkylaminocarbonyl;
Het is a monocyclic heterocycle selected from the group of N-phenoxypiperidinyl, pyrrolyl, pyrazolyl, imidazolyl, furanyl, thienyl, oxazolyl, isoxazolyl, thiazolyl, isothiazolyl, pyridinyl, pyrimidinyl, pyrazinyl and pyridazinyl; or a bicyclic heterocycle selected from the group of quinolinyl, quinoxalinyl, indolyl, benzimidazolyl, benzoxazolyl, benzisoxazolyl, benzothiazolyl, benzisothiazolyl, benzofuranyl, benzothienyl, 2,3-dihydrobenzo[1,4]dioxinyl or benzo[1,3]dioxolyl; each monocyclic and bicyclic heterocycle may optionally be substituted on a carbon atom with 1, 2 or 3 substituents selected from the group of halo, hydroxy, alkyl or alkyloxy;
halo is a substituent selected from the group of fluoro, chloro, bromo and iodo and
haloalkyl is a straight or branched saturated hydrocarbon radical having from 1 to 6 carbon atoms or a cyclic saturated hydrocarbon radical having from 3 to 6 carbon atoms, wherein one or more carbon atoms are substituted with one or more halo-atoms.

The invention also relates to the use as defined hereinabove of compounds of Formula (Ia) or (Ib) wherein:
R¹ is hydrogen, halo, cyano, Ar, Het, alkyl, and alkyloxy;
p is an integer equal to zero, 1, 2, 3 or 4;
R² is hydrogen, hydroxy, alkyloxy, alkyloxyalkyloxy, alkylthio or a radical of formula 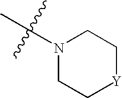
   wherein Y is 0;
R³ is alkyl, Ar, Ar-alkyl or Het;
q is an integer equal to zero, 1, 2, or 3;
R⁴ and R⁵ each independently are hydrogen, alkyl or benzyl; or
R⁴ and R⁵ together and including the N to which they are attached may form a radical selected from the group of pyrrolidinyl, imidazolyl, triazolyl, piperidinyl, piperazinyl, pyrazinyl, morpholinyl and thiomorpholinyl, optionally substituted with alkyl and pyrimidinyl;
R⁶ is hydrogen, halo or alkyl; or
two vicinal R⁶ radicals may be taken together to form a bivalent radical of formula —CH═CH—CH═CH—;
r is an integer equal to 1; and
R⁷ is hydrogen;
R⁸ is hydrogen or alkyl;
R⁹ is oxo; or
R⁸ and R⁹ together form the radical ═N—CH═CH—.
alkyl is a straight or branched saturated hydrocarbon radical having from 1 to 6 carbon atoms; or is a cyclic saturated hydrocarbon radical having from 3 to 6 carbon atoms; or is a a cyclic saturated hydrocarbon radical having from 3 to 6 carbon atoms attached to a straight or branched saturated hydrocarbon radical having from 1 to 6 carbon atoms; wherein each carbon atom can be optionally substituted with halo or hydroxy;
Ar is a homocycle selected from the group of phenyl, naphthyl, acenaphthyl, tetrahydronaphthyl, each optionally substituted with 1, 2 or 3 substituents, each substituent independently selected from the group of halo, haloalkyl, cyano, alkyloxy and morpholinyl;
Het is a monocyclic heterocycle selected from the group of N-phenoxypiperidinyl, furanyl, thienyl, pyridinyl, pyrimidinyl; or a bicyclic heterocycle selected from the group of benzothienyl, 2,3-dihydrobenzo[1,4]dioxinyl or benzo[1,3]-dioxolyl; each monocyclic and bicyclic heterocycle may optionally be substituted on a carbon atom with 1, 2 or 3 alkyl substituents; and
halo is a substituent selected from the group of fluoro, chloro and bromo.

For compounds according to either Formula (Ia) and (Ib), preferably, R¹ is hydrogen, halo, Ar, alkyl or alkyloxy. More preferably, R¹ is halo. Most preferably, R¹ is bromo.

Preferably, p is equal to 1.

Preferably, R² is hydrogen, alkyloxy or alkylthio. More preferably, R² is alkyloxy, in particular C₁₋₄alkyloxy. Most preferably, R² is methyloxy.

C₁₋₄alkyl is a straight or branched saturated hydrocarbon radical having from 1 to 4 carbon atoms such as for example methyl, ethyl, propyl, 2-methyl-ethyl and the like.

Preferably, R³ is naphthyl, phenyl or thienyl, each optionally substituted with 1 or 2 substituents, that substituent preferably being a halo or haloalkyl, most preferably being a halo. More preferably, R³ is naphthyl or phenyl, each optionally substituted with halo, preferably 3-fluoro. Even more preferably, R³ is naphthyl or phenyl. Most preferably, R³ is naphthyl.

Preferably, q is equal to zero, 1 or 2. More preferably, q is equal to 1.

Preferably, R⁴ and R⁵ each independently are hydrogen or alkyl, in particular hydrogen or C₁₋₄alkyl, more in particular C₁₋₄alkyl, more preferably hydrogen, methyl or ethyl, most preferably methyl.

C₁₋₄alkyl is a straight or branched saturated hydrocarbon radical having from 1 to 4 carbon atoms such as for example methyl, ethyl, propyl, 2-methyl-ethyl and the like.

Preferably R⁴ and R⁵ together and including the N to which they are attached form a radical selected from the group of imidazolyl, triazolyl, piperidinyl, piperazinyl and thiomorpholinyl, optionally substituted with alkyl, halo, haloalkyl, hydroxy, alkyloxy, alkylthio, alkyloxyalkyl or alkylthioalkyl, preferably substituted with alkyl, most preferably substituted with methyl or ethyl.

Preferably, R⁶ is hydrogen, alkyl or halo. Most preferably, R⁶ is hydrogen. Preferably r is 0, 1 or 2.

Preferably, R⁷ is hydrogen or methyl, more preferably hydrogen.

For compounds according to Formula (Ib) only, preferably, R⁸ is alkyl, preferably methyl and R⁹ is oxygen.

An interesting group of compounds are the compounds according to formula (Ia), the pharmaceutically acceptable acid or base addition salts thereof, the stereochemically isomeric forms thereof, the tautomeric forms thereof or the N-oxide forms thereof

An interesting group of compounds are the compounds according to Formula (Ia), the pharmaceutically acceptable acid or base addition salts thereof, the stereochemically

1-(6-bromo-2-methoxy-quinolin-3-yl)-4-dimethylamino-2-naphthalen-1-yl-1-phenyl-butan-2-ol corresponding to 6-bromo-α-[2-(dimethylamino)ethyl]-2-methoxy-α-1-naphthalenyl-β-phenyl-3-quinolineethanol;

a pharmaceutically acceptable acid or base addition salt thereof, a stereochemically isomeric form thereof, a tautomeric form thereof or a N-oxide form thereof.

An alternative chemical name for 1-(6-bromo-2-methoxy-quinolin-3-yl)-4-dimethylamino-2-naphthalen-1-yl-1-phenyl-butan-2-ol is 6-bromo-α-[2-(dimethylamino)ethyl]-2-methoxy-α-1-naphthalenyl-β-phenyl-3-quinolineethanol. Said compound can also be represented as follows:

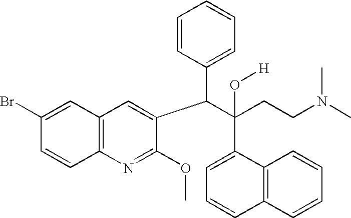

Most preferably, the compound is one of the following:

6-bromo-α-[2-(dimethylamino)ethyl]-2-methoxy-α-1-naphthalenyl-β-phenyl-3-quinolineethanol, a pharmaceutically acceptable acid or base addition salt thereof, a stereochemically isomeric forms thereof, a tautomeric form thereof or a N-oxide form thereof; or

6-bromo-α-[2-(dimethylamino)ethyl]-2-methoxy-α-1-naphthalenyl-β-phenyl-3-quinolineethanol, or a pharmaceutically acceptable acid addition salt thereof; or

6-bromo-α-[2-(dimethylamino)ethyl]-2-methoxy-α-1-naphthalenyl-β-phenyl-3-quinolineethanol, or a stereochemically isomeric form thereof; or

6-bromo-α-[2-(dimethylamino)ethyl]-2-methoxy-α-1-naphthalenyl-β-phenyl-3-quinolineethanol, or a N-oxide form thereof; or

(αS, βR)-6-bromo-α-[2-(dimethylamino)ethyl]-2-methoxy-α-1-naphthalenyl-β-phenyl-3-quinolineethanol, i.e. compound 12, or a pharmaceutically acceptable acid addition salt thereof; or

(αS, βR)-6-bromo-α-[2-(dimethylamino)ethyl]-2-methoxy-α-1-naphthalenyl-β-phenyl-3-quinolineethanol, i.e. compound 12.

isomeric forms thereof, the tautomeric forms thereof or the N-oxide forms thereof, in which R¹ is hydrogen, halo, Ar, alkyl or alkyloxy, p=1, R² is hydrogen, alkyloxy or alkylthio, R³ is naphthyl, phenyl or thienyl, each optionally substituted with 1 or 2 substituents selected from the group of halo and haloalkyl, q=0, 1, 2 or 3, R⁴ and R⁵ each independently are hydrogen or alkyl or R⁴ and R⁵ together and including the N to which they are attached form a radical selected from the group of imidazolyl, triazolyl, piperidinyl, piperazinyl and thiomorpholinyl, R⁶ is hydrogen, alkyl or halo, r is equal to 0 or 1 and R⁷ is hydrogen.

Preferable, the compound is:

1-(6-bromo-2-methoxy-quinolin-3-yl)-2-(3,5-difluoro-phenyl)-4-dimethylamino-1-phenyl-butan-2-ol;

1-(6-bromo-2-methoxy-quinolin-3-yl)-4-dimethylamino-2-naphthalen-1-yl-1-phenyl-butan-2-ol corresponding to 6-bromo-α-[2-(dimethylamino)ethyl]-2-methoxy-α-1-naphthalenyl-β-phenyl-3-quinolineethanol;

1-(6-bromo-2-methoxy-quinolin-3-yl)-2-(2,5-difluoro-phenyl)-4-dimethylamino-1-phenyl-butan-2-ol;

1-(6-bromo-2-methoxy-quinolin-3-yl)-2-(2,3-difluoro-phenyl)-4-dimethylamino-1-phenyl-butan-2-ol;

1-(6-bromo-2-methoxy-quinolin-3-yl)-4-dimethylamino-2-(2-fluoro-phenyl)-1-phenyl-butan-2-ol;

1-(6-bromo-2-methoxy-quinolin-3-yl)-4-dimethylamino-2-naphthalen-1-yl-1-p-tolyl-butan-2-ol;

1-(6-bromo-2-methoxy-quinolin-3-yl)-4-methylamino-2-naphthalen-1-yl-1-phenyl-butan-2-ol;

1-(6-bromo-2-methoxy-quinolin-3-yl)-4-dimethylamino-2-(3-fluoro-phenyl)-1-phenyl-butan-2-ol; and

1-(6-bromo-2-methoxy-quinolin-3-yl)-4-dimethylamino-2-phenyl-1-phenyl-butan-2-ol;

a pharmaceutically acceptable acid or base addition salt thereof, a stereochemically isomeric form thereof, a tautomeric form thereof or a N-oxide form thereof.

Even more preferably, the compound is

1-(6-bromo-2-methoxy-quinolin-3-yl)-4-dimethylamino-2-(3-fluoro-phenyl)-1-phenyl-butan-2-ol;

1-(6-bromo-2-methoxy-quinolin-3-yl)-4-dimethylamino-2-phenyl-1-phenyl-butan-2-ol;

Thus, most preferably, the compound is (αS, βR)-6-bromo-α-[2-(dimethylamino)ethyl]-2-methoxy-α-1-naphthalenyl-β-phenyl-3-quinolineethanol which corresponds to (1R,2S)-1-(6-bromo-2-methoxy-quinolin-3-yl)-4-dimethylamino-2-naphthalen-1-yl-1-phenyl-butan-2-ol. Said compound can also be represented as follows:

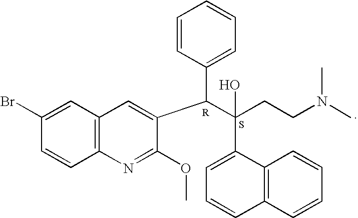

The pharmaceutically acceptable acid addition salts are defined to comprise the therapeutically active non-toxic acid addition salt forms which the compounds according to either Formula (Ia) and (Ib) are able to form. Said acid addition salts can be obtained by treating the base form of the compounds according to either Formula (Ia) and (Ib) with appropriate acids, for example inorganic acids, for example hydrohalic acid, in particular hydrochloric acid, hydrobromic acid, sulfuric acid, nitric acid and phosphoric acid; organic acids, for example acetic acid, hydroxyacetic acid, propanoic acid, lactic acid, pyruvic acid, oxalic acid, malonic acid, succinic acid, maleic acid, fumaric acid, malic acid, tartaric acid, citric acid, methanesulfonic acid, ethanesulfonic acid, benzenesulfonic acid, p-toluenesulfonic acid, cyclamic acid, salicyclic acid, p-aminosalicylic acid and pamoic acid.

The compounds according to either Formula (Ia) and (Ib) containing acidic protons may also be converted into their therapeutically active non-toxic base addition salt forms by treatment with appropriate organic and inorganic bases. Appropriate base salts forms comprise, for example, the ammonium salts, the alkaline and earth alkaline metal salts, in particular lithium, sodium, potassium, magnesium and calcium salts, salts with organic bases, e.g. the benzathine, N-methyl-D-glucamine, hybramine salts, and salts with amino acids, for example arginine and lysine.

Conversely, said acid or base addition salt forms can be converted into the free forms by treatment with an appropriate base or acid.

The term addition salt as used in the framework of this application also comprises the solvates which the compounds according to either Formula (Ia) and (Ib) as well as the salts thereof, are able to form. Such solvates are, for example, hydrates and alcoholates.

The term “stereochemically isomeric forms” as used herein defines all possible isomeric forms which the compounds of either Formula (Ia) and (Ib) may possess. Unless otherwise mentioned or indicated, the chemical designation of compounds denotes the mixture of all possible stereochemically isomeric forms, said mixtures containing all diastereomers and enantiomers of the basic molecular structure. More in particular, stereogenic centers may have the R- or S-configuration; substituents on bivalent cyclic (partially) saturated radicals may have either the cis- or trans-configuration. Stereochemically isomeric forms of the compounds of either Formula (Ia) and (Ib) are obviously intended to be embraced within the scope of this invention.

Following CAS-nomenclature conventions, when two stereogenic centers of known absolute configuration are present in a molecule, an R or S descriptor is assigned (based on Cahn-Ingold-Prelog sequence rule) to the lowest-numbered chiral center, the reference center. The configuration of the second stereogenic center is indicated using relative descriptors [R*,R*] or [R*,S*], where R* is always specified as the reference center and [R*,R*] indicates centers with the same chirality and [R*,S*] indicates centers of unlike chirality. For example, if the lowest-numbered chiral center in the molecule has an S configuration and the second center is R, the stereo descriptor would be specified as S-[R*,S*]. If “α” and “β” are used: the position of the highest priority substituent on the asymmetric carbon atom in the ring system having the lowest ring number, is arbitrarily always in the “α” position of the mean plane determined by the ring system. The position of the highest priority substituent on the other asymmetric carbon atom in the ring system relative to the position of the highest priority substituent on the reference atom is denominated “α”, if it is on the same side of the mean plane determined by the ring system, or “β”, if it is on the other side of the mean plane determined by the ring system.

Compounds of either Formula (Ia) and (Ib) and some of the intermediate compounds invariably have at least two stereogenic centers in their structure which may lead to at least 4 stereochemically different structures.

The tautomeric forms of the compounds of either Formula (Ia) and (Ib) are meant to comprise those compounds of either Formula (Ia) and (Ib) wherein e.g. an enol group is converted into a keto group (keto-enol tautomerism).

The N-oxide forms of the compounds according to either Formula (Ia) and (Ib) are meant to comprise those compounds of either Formula (Ia) and (Ib) wherein one or several tertiary nitrogen atoms are oxidized to the so-called N-oxide.

The compounds of either Formula (Ia) and (Ib) as prepared in the processes described below may be synthesized in the form of racemic mixtures of enantiomers which can be separated from one another following art-known resolution procedures. The racemic compounds of either Formula (Ia) and (Ib) may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of either Formula (Ia) and (Ib) involves liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound will be synthesized by stereospecific methods of preparation. These methods will advantageously employ enantiomerically pure starting materials.

The invention also comprises derivative compounds (usually called “pro-drugs”) of the pharmacologically-active compounds according to the invention, which are degraded in vivo to yield the compounds according to the invention. Pro-drugs are usually (but not always) of lower potency at the target receptor than the compounds to which they are degraded. Pro-drugs are particularly useful when the desired compound has chemical or physical properties that make its administration difficult or inefficient. For example, the desired compound may be only poorly soluble, it may be poorly transported across the mucosal epithelium, or it may have an undesirably short plasma half-life. Further discussion on pro-drugs may be found in Stella, V. J. et al., “Prodrugs”, Drug Delivery Systems, 1985, pp. 112-176, and Drugs, 1985, 29, pp. 455-473.

Pro-drugs forms of the pharmacologically-active compounds according to the invention will generally be compounds according to either Formula (Ia) and (Ib), the pharmaceutically acceptable acid or base addition salts thereof, the stereochemically isomeric forms thereof, the tautomeric forms thereof and the N-oxide forms thereof, having an acid group which is esterified or amidated. Included in such esterified acid groups are groups of the formula —COOR^{x}, where R^{x} is a C₁₋₆alkyl, phenyl, benzyl or one of the following groups:

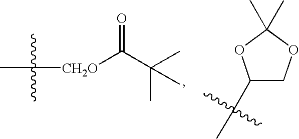

Amidated groups include groups of the formula —CONR^{y}R^{z}, wherein R^{y} is H, C₁₋₆alkyl, phenyl or benzyl and R^{z} is —OH, H, C₁₋₆alkyl, phenyl or benzyl.

Compounds according to the invention having an amino group may be derivatised with a ketone or an aldehyde such as formaldehyde to form a Mannich base. This base will hydrolyze with first order kinetics in aqueous solution.

An interesting embodiment of the present invention is the use of a substituted quinoline derivative according to Formula (Ia) or Formula (Ib), in particular (αS, βR)-6-bromo-α-[2-(dimethylamino)ethyl]-2-methoxy-α-1-naphthalenyl-β-phenyl-3-quinolineethanol, for the preparation of a medicament for the treatment of an infection with a drug resistant Mycobacterium strain as defined hereinabove wherein the drug resistant Mycobacterium strain is a drug resistant M. tuberculosis strain.

A further interesting embodiment of the present invention is the use of a substituted quinoline derivative according to Formula (Ia) or Formula (Ib), in particular (αS, βR)-6-bromo-α-[2-(dimethylamino)ethyl]-2-methoxy-α-1-naphthalenyl-β-phenyl-3-quinolineethanol, for the preparation of a medicament for the treatment of a human infected with a drug resistant Mycobacterium strain, in particular a drug resistant M. tuberculosis strain.

Still a further interesting embodiment of the present invention is the use of a substituted quinoline derivative according to Formula (Ia) or Formula (Ib), in particular (αS, βR)-6-bromo-α-[2-(dimethylamino)ethyl]-2-methoxy-α-1-naphthalenyl-β-phenyl-3-quinolineethanol, for the preparation of a medicament for the treatment of an infection with a multi drug resistant Mycobacterium strain, in particular a multi drug resistant M. tuberculosis strain, in particular for the preparation of a medicament for the treatment of a mammal, including a human, infected with a multi drug resistant Mycobacterium strain, in particular a multi drug resistant M. tuberculosis strain.

As already stated above, the compounds of formula (Ia) and (Ib) can be used to treat drug resistant including multi drug resistant Mycobacterial diseases. The exact dosage and frequency of administration depends on the particular compound of formula (Ia) or (Ib) used, the particular condition being treated, the severity of the condition being treated, the age, weight and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

Given the fact that the compounds of formula (Ia) and (Ib) are active against drug resistant including multi drug resistant Mycobacterial strains, the present compounds may be combined with other antimycobacterial agents in order to effectively combat Mycobacterial diseases.

Therefore, the present invention also relates to a combination of (a) a compound of formula (Ia) or (Ib), in particular (αS, βR)-6-bromo-α-[2-(dimethylamino)ethyl]-2-methoxy-α-1-naphthalenyl-β-phenyl-3-quinolineethanol or a pharmaceutically acceptable acid addition salt thereof, and (b) one or more other antimycobacterial agents.

The present invention also relates to a combination of (a) a compound of formula (Ia) or (Ib), in particular (αS, βR)-6-bromo-α-[2-(dimethylamino)ethyl]-2-methoxy-α-1-naphthalenyl-β-phenyl-3-quinolineethanol or a pharmaceutically acceptable acid addition salt thereof, and (b) one or more other antimycobacterial agents for use as a medicine.

A pharmaceutical composition comprising a pharmaceutically acceptable carrier and, as active ingredient, a therapeutically effective amount of (a) a compound of formula (Ia) or (Ib), in particular (αS, βR)-6-bromo-α-[2-(dimethylamino)ethyl]-2-methoxy-α-1-naphthalenyl-β-phenyl-3-quinolineethanol or a pharmaceutically acceptable acid addition salt thereof, and (b) one or more other antimycobacterial agents, is also comprised by the present invention.

The present invention also relates to the use of a combination or pharmaceutical composition as defined above for the treatment of an infection with a drug resistant Mycobacterium strain, in particular a drug resistant M tuberculosis strain. The above defined combination or pharmaceutical composition may also be used to treat an infection with a susceptible Mycobacterial strain, in particular a susceptible M tuberculosis strain.

In the above defined combination or pharmaceutical composition, the compound of formula (Ia) or (Ib) is preferably a compound of formula (Ia).

The other Mycobacterial agents which may be combined with the compounds of formula (Ia) or (Ib) are for example rifampicin (=rifampin); isoniazid; pyrazinamide; amikacin; ethionamide; moxifloxacin; ethambutol; streptomycin; para-aminosalicylic acid; cycloserine; capreomycin; kanamycin; thioacetazone; PA-824; quinolones/fluoroquinolones such as for example ofloxacin, ciprofloxacin, sparfloxacin; macrolides such as for example clarithromycin, clofazimine, amoxycillin with clavulanic acid; rifamycins; rifabutin; rifapentine.

Preferably, the present compounds of formula (Ia) or (Ib), in particular (αS, βR)-6-bromo-α-[2-(dimethylamino)ethyl]-2-methoxy-α-1-naphthalenyl-β-phenyl-3-quinolineethanol, are combined with rifapentin and moxifloxacin.

Another interesting combination according to the present invention is a combination of (a) a compound of formula (Ia) or (Ib), in particular (αS, βR)-6-bromo-α-[2-(dimethylamino)ethyl]-2-methoxy-α-1-naphthalenyl-β-phenyl-3-quinolineethanol or a pharmaceutically acceptable acid addition salt thereof, and (b) one or more other antimycobacterial agents wherein said one or more other antimycobacterial agents comprise pyrazinamide. Thus, the present invention also relates to a combination of a compound of formula (Ia) or (Ib), in particular (αS, βR)-6-bromo-α-[2-(dimethylamino)ethyl]-2-methoxy-α-1-naphthalenyl-β-phenyl-3-quinolineethanol or a pharmaceutically acceptable acid addition salt thereof, and pyrazinamide and optionally one or more other antimycobacterial agents. Examples of such combinations are the combination of (αS, βR)-6-bromo-α-[2-(dimethylamino)ethyl]-2-methoxy-α-1-naphthalenyl-β-phenyl-3-quinolineethanol or a pharmaceutically acceptable acid addition salt thereof, and pyrazinamide; the combination of (αS, βR)-6-bromo-α-[2-(dimethylamino)ethyl]-2-methoxy-α-1-naphthalenyl-β-phenyl-3-quinolineethanol or a pharmaceutically acceptable acid addition salt thereof, pyrazinamide and rifapentin; the combination of (αS, βR)-6-bromo-α-[2-(dimethylamino)ethyl]-2-methoxy-α-1-naphthalenyl-β-phenyl-3-quinolineethanol or a pharmaceutically acceptable acid addition salt thereof, pyrazinamide and isoniazid; the combination of (αS, βR)-6-bromo-α-[2-(dimethylamino)ethyl]-2-methoxy-α-1-naphthalenyl-β-phenyl-3-quinolineethanol or a pharmaceutically acceptable acid addition salt thereof, pyrazinamide and moxifloxacin; the combination of (αS, βR)-6-bromo-α-[2-(dimethylamino)ethyl]-2-methoxy-α-1-naphthalenyl-β-phenyl-3-quinolineethanol or a pharmaceutically acceptable acid addition salt thereof, pyrazinamide and rifampin. It has been found that a compound of formula (Ia) or (Ib), in particular (αS, βR)-6-bromo-α-[2-(dimethylamino)ethyl]-2-methoxy-α-1-naphthalenyl-β-phenyl-3-quinolineethanol or a pharmaceutically acceptable acid addition salt thereof, and pyrazinamide act synergistically.

Also interesting combinations are those combinations comprising a compound of formula (Ia) or (Ib), as described in Tables 11 and 12.

A pharmaceutical composition comprising a pharmaceutically acceptable carrier and, as active ingredient, a therapeutically effective amount of the active ingredients listed in the above combinations, is also comprised by the present invention.

The present pharmaceutical composition may have various pharmaceutical forms for administration purposes. As appropriate compositions there may be cited all compositions usually employed for systemically administering drugs. To prepare the pharmaceutical compositions of this invention, an effective amount of the particular compounds, optionally in addition salt form, as the active ingredient is combined in intimate admixture with a pharmaceutically acceptable carrier, which carrier may take a wide variety of forms depending on the form of preparation desired for administration. These pharmaceutical compositions are desirable in unitary dosage form suitable, in particular, for administration orally or by parenteral injection. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, diluents, lubricants, binders, disintegrating agents and the like in the case of powders, pills, capsules and tablets. Because of their ease in administration, tablets and capsules represent the most advantageous oral unit dosage forms in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations which are intended to be converted, shortly before use, to liquid form preparations.

Depending on the mode of administration, the pharmaceutical composition will preferably comprise from 0.05 to 99% by weight, more preferably from 0.1 to 70% by weight of the active ingredients, and, from 1 to 99.95% by weight, more preferably from 30 to 99.9 weight % of a pharmaceutically acceptable carrier, all percentages being based on the total composition.

The weight to weight ratio's of the compound of formula (Ia) or (Ib) and (b) the other antimycobacterial agent(s) when given as a combination may be determined by the person skilled in the art. Said ratio and the exact dosage and frequency of administration depends on the particular compound of formula (Ia) or (Ib) and the other antimycobacterial agent(s) used, the particular condition being treated, the severity of the condition being treated, the age, weight and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

The compounds of formula (Ia) or (Ib) and the one or more other antimycobacterial agents may be combined in a single preparation or they may be formulated in separate preparations so that they can be administered simultaneously, separately or sequentially. Thus, the present invention also relates to a product containing (a) a compound of formula (Ia) or (Ib), and (b) one or more other antimycobacterial agents, as a combined preparation for simultaneous, separate or sequential use in the treatment of mycobacterial diseases.

The pharmaceutical composition may additionally contain various other ingredients known in the art, for example, a lubricant, stabilising agent, buffering agent, emulsifying agent, viscosity-regulating agent, surfactant, preservative, flavouring or colorant.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, powder packets, wafers, suppositories, injectable solutions or suspensions and the like, and segregated multiples thereof. The daily dosage of the compound according to the invention will, of course, vary with the compound employed, the mode of administration, the treatment desired and the mycobacterial disease indicated. However, in general, satisfactory results will be obtained when the compound according to the invention is administered at a daily dosage not exceeding 1 gram, e.g. in the range from 10 to 50 mg/kg body weight.

The compounds of formula (Ia) and (Ib) and their preparation is described in WO 2004/011436, which is incorporated herein by reference.

Of some compounds the absolute stereochemical configuration of the stereogenic carbon atom(s) therein was not experimentally determined. In those cases the stereochemically isomeric form which was first isolated is designated as “A” and the second as “B”, without further reference to the actual stereochemical configuration. However, said “A” and “B” isomeric forms can be unambiguously characterized by a person skilled in the art, using art-known methods such as, for example, X-ray diffraction.

In case “A” and “B” are stereoisomeric mixtures, they can be further separated whereby the respective first fractions isolated are designated “A1” and “B1” and the second as “A2” and “B2”, without further reference to the actual stereochemical configuration.

The following Tables list compounds of formula (Ia) and (Ib), which can all be prepared according to the methods described in WO 2004/011436.

TABLE 1

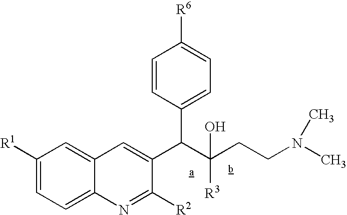

| | | | | | | Stereochemistry and melting |
|---|---|---|---|---|---|---|
| Comp. nr. | Ex. nr. | R¹ | R² | R³ | R⁶ | points |
| 1 | B1 | Br | OCH₃ | phenyl | H | (A1); 194° C. |
| 2 | B1 | Br | OCH₃ | phenyl | H | (A2); 191° C. |
| 3 | B1 | Br | OCH₃ | phenyl | H | (A); 200° C. |
| 4 | B1 | Br | OCH₃ | phenyl | H | (B); 190° C. |
| 16 | B1 | Br | OCH₃ | 4-chlorophenyl | H | (A); 200° C. |
| 17 | B1 | Br | OCH₃ | 4-chlorophenyl | H | (B); 190° C. |
| 20 | B1 | Br | OCH₃ | 2-thienyl | H | (A); 96° C. |
| 21 | B1 | Br | OCH₃ | 2-thienyl | H | (B); 176° C. |
| 22 | B1 | CH₃ | OCH₃ | phenyl | H | (A); 148° C. |
| 23 | B1 | CH₃ | OCH₃ | phenyl | H | (B); 165° C. |
| 24 | B1 | Br | OCH₃ | 3-thienyl | H | (A); 162° C. |
| 25 | B1 | Br | OCH₃ | 3-thienyl | H | (B); 160° C. |
| 26 | B1 | phenyl | OCH₃ | phenyl | H | (A); 174° C. |
| 27 | B1 | phenyl | OCH₃ | phenyl | H | (B); 192° C. |
| 28 | B1 | F | OCH₃ | phenyl | H | (A); 190° C. |
| 29 | B1 | F | OCH₃ | phenyl | H | (B); 166° C. |
| 30 | B1 | Cl | OCH₃ | phenyl | H | (A); 170° C. |
| 31 | B1 | Cl | OCH₃ | phenyl | H | (B); 181° C. |
| 32 | B1 | Br | SCH₃ | phenyl | H | (A); 208° C. |
| 33 | B1 | Br | SCH₃ | phenyl | H | (B); 196° C. |
| 34 | B1 | OCH₃ | OCH₃ | phenyl | H | (A); 165° C. |
| 35 | B1 | OCH₃ | OCH₃ | phenyl | H | (B); 165° C. |
| 36 | B1 | Br | OCH₃ | phenyl | Cl | (A); 197° C. |
| 37 | B1 | Br | OCH₃ | phenyl | Cl | (B); 221° C. |
| 38 | B9 | Br | OCH₃ | 3-fluorophenyl | H | (A); 198° C. |
| 39 | B9 | Br | OCH₃ | 3-fluorophenyl | H | (B); 207° C. |
| 108 | B9 | Br | OCH₃ | 3-fluorophenyl | H | (A1); 160° C. |
| 109 | B9 | Br | OCH₃ | 3-fluorophenyl | H | (A2); 156° C. |
| 40 | B1 | H | OCH₃ | phenyl | H | (A); 152° C. |
| 41 | B1 | H | OCH₃ | phenyl | H | (B); 160° C. |
| 42 | B1 | H | OCH₃ | CH₃ | H | (A); 140° C. |
| 43 | B1 | H | OCH₃ | CH₃ | H | (B); 120° C. |
| 59 | B1 | Br | OH | phenyl | H | (A); >260° C. |
| 60 | B1 | Br | OH | phenyl | H | (B); 215° C. |
| 5 | B2 | Br | OCH₂CH₃ | phenyl | H | (A); 162° C. |
| 6 | B2 | Br | OCH₂CH₃ | phenyl | H | (B); 74° C. |
| 7 | B3 | Br | H | phenyl | H | (A); 98° C. |
| 8 | B3 | Br | H | phenyl | H | (B); 180° C. |
| 12 | B7 | Br | OCH₃ | 1-naphthyl | H | (A1); 118° C.; a = R, b = S; |
| | | | | | | [alpha]_{D}²⁰ = −166.98 |
| | | | | | | (c = 0.505 g/100 ml in DMF) |
| 13 | B7 | Br | OCH₃ | 1-naphthyl | H | (A2); 120° C.; a = S; b = R; |
| | | | | | | [alpha]_{D}²⁰ = +167.60 |
| | | | | | | (c = 0.472 g/100 ml in DMF) |
| 14 | B7 | Br | OCH₃ | 1-naphthyl | H | (A); 210° C. |
| 15 | B7 | Br | OCH₃ | 1-naphthyl | H | (B); 244° C. |
| 45 | B7 | Br | OCH₃ | 2-naphthyl | H | (A); 262° C. |
| 46 | B7 | Br | OCH₃ | 2-naphthyl | H | (B); 162° C. |
| 67 | B8 | Br | OCH₃ | 2,5-difluorophenyl | H | (A); 60° C. |
| 68 | B8 | Br | OCH₃ | 2,5-difluorophenyl | H | (B); 208° C. |
| 110 | B8 | Br | OCH₃ | 2,5-difluorophenyl | H | (A1); 167° C. |
| 111 | B8 | Br | OCH₃ | 2,5-difluorophenyl | H | (A2); oil |
| 69 | B1 | Br | OCH₃ | 2-fluorophenyl | H | (A); oil |
| 70 | B1 | Br | OCH₃ | 2-fluorophenyl | H | (B); oil |
| 71 | B1 | Br | OCH₃ | 1-naphthyl | CH₃ | (A); 174° C. |
| 72 | B1 | Br | OCH₃ | 1-naphthyl | CH₃ | (B); 178° C. |
| 73 | B1 | Br | OCH₃ | 1-naphthyl | Cl | (B); 174° C. |
| 74 | B1 | Br | OCH₃ | 1-naphthyl | Cl | (A); 110° C. |
| 75 | B1 | Br | OCH₃ | 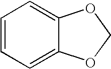 | H | (A); 196° C. |
| 76 | B1 | Br | OCH₃ | 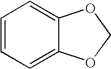 | H | (B); 130° C. |
| 77 | B1 | Br | OCH₃ | 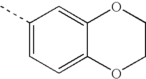 | H | (A); 202° C. |
| 78 | B1 | Br | OCH₃ | 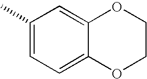 | H | (B); 202° C. |
| 79 | B1 | Br | 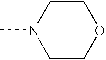 | 1-naphthyl | H | (A); >250° C. |
| 80 | B1 | Br | OCH₃ | 4-cyanophenyl | H | (A); 224° C. |
| 81 | B1 | Br | OCH₃ | 4-cyanophenyl | H | (B); 232° C. |
| 82 | B1 | CH₃ | OCH₃ | 1-naphthyl | H | (A); 202° C. |
| 83 | B1 | CH₃ | OCH₃ | 1-naphthyl | H | (B); 198° C. |
| 84 | B1 | phenyl | OCH₃ | 1-naphthyl | H | (A); 248° C. |
| 85 | B1 | phenyl | OCH₃ | 1-naphthyl | H | (B); 214° C. |
| 86 | B1 | Br | OCH₃ | 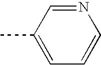 | H | (A); 184° C. |
| 87 | B1 | Br | OCH₃ | 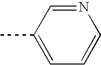 | H | (B); 186° C. |
| 88 | B1 | Br | SCH₃ | 1-naphthyl | H | (A); 240° C. |
| 89 | B1 | Br | OCH₃ | 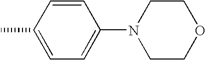 | H | (A); 236° C. |
| 90 | B1 | Br | OCH₃ | 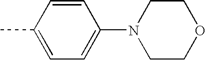 | H | (B); 206° C. |
| 91 | B1 | H | OCH₃ | 1-naphthyl | H | (A); 178° C. |
| 92 | B1 | H | OCH₃ | 1-naphthyl | H | (B); 160° C. |
| 93 | B1 | H | OCH₃ | 3-fluorophenyl | H | (A); 178° C. |
| 94 | B1 | H | OCH₃ | 3-fluorophenyl | H | (B); 182° C. |
| 95 | B1 | Br | OCH₃ | 2-phenylethyl | H | (A); 178° C. |
| 96 | B1 | Br | OCH₃ | 2-phenylethyl | H | (B); 146° C. |
| 97 | B1 | OCH₃ | OCH₃ | 1-naphthyl | H | (A); 168° C. |
| 98 | B1 | OCH₃ | OCH₃ | 1-naphthyl | H | (B); 154° C. |
| 113 | B14 | Br | OCH₃ | 2,3-difluorophenyl | H | (A); 128° C. |
| 114 | B14 | Br | OCH₃ | 2,3-difluorophenyl | H | (B); 213° C. |
| 115 | B15 | Br | OCH₃ | 3,5-difluorophenyl | H | (A); 192° C. |
| 116 | B15 | Br | OCH₃ | 3,5-difluorophenyl | H | (B); 224° C. |
| 117 | B15 | Br | OCH₃ | 3,5-difluorophenyl | H | (A1); 161° C. |
| 118 | B15 | Br | OCH₃ | 3,5-difluorophenyl | H | (A2); 158° C. |
| 119 | B7 | Cl | OCH₃ | 1-naphthyl | H | (A); 212° C. |
| 120 | B7 | Cl | OCH₃ | 1-naphthyl | H | (B); 236° C. |
| 122 | B7 | Br | OCH₃ | 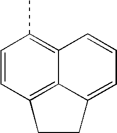 | H | (B); 227° C. |
| 127 | B7 | Br | OCH₃ | 5-bromo-2-naphthyl | H | (A); 226° C. |
| 130 | B7 | Br | OCH₃ | 5-bromo-2-naphthyl | H | (B); 220° C. |
| 131 | B1 | Br | OCH₃ | 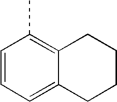 | H | (A); 206° C. |
| 134 | B9 | OCH₃ | OCH₃ | 3-fluorophenyl | H | (A); 172° C. |
| 135 | B9 | OCH₃ | OCH₃ | 3-fluorophenyl | H | (B); 182° C. |
| 143 | B7 | Br | OCH₃ | 3-bromo-1-naphthyl | H | (A); 234° C. |
| 150 | B7 | Br | OCH₃ | 3-bromo-1-naphthyl | H | (B); 212° C. |
| 159 | B8 | Br | OCH₃ | 2,5-difluorophenyl | H | (A1); 208° C. |
| 160 | B8 | Br | OCH₃ | 2,5-difluorophenyl | H | (A2); 167° C. |
| 162 | B7 | Br | OCH₃ | 6-methoxy-2-naphthyl | H | (A); 206° C. |
| 163 | B7 | Br | OCH₃ | 6-methoxy-2-naphthyl | H | (B); 206° C. |
| 164 | B9 | Br | 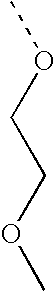 | 3-fluorophenyl | H | (A); 118° C. |
| 165 | B9 | Br | 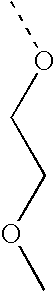 | 3-fluorophenyl | H | (B); oil |
| 167 | B8 | Br | OCH₃ | 2,6-difluorophenyl | H | (B); 180° C. |
| 174 | B9 | 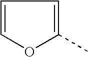 | OCH₃ | 3-fluorophenyl | H | (A); 159° C. |
| 175 | B9 | 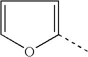 | OCH₃ | 3-fluorophenyl | H | (B); 196° C. |
| 176 | B7 | Br | 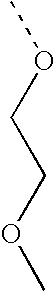 | 1-naphthyl | H | (A); oil |
| 179 | B9 | CN | OCH₃ | 3-fluorophenyl | H | (A); 213° C. |
| 180 | B9 | CN | OCH₃ | 3-fluorophenyl | H | (B); 163° C. |
| 181 | B9 | Br | OCH₃ | 4-fluorophenyl | H | (A); 198° C. |
| 182 | B9 | Br | OCH₃ | 4-fluorophenyl | H | (B); 238° C. |
| 183 | B1 | Br | OCH₃ | 3-trifluoro- | H | (A); 170° C. |
| | | | | methylphenyl | | |
| 188 | B1 | Br | OCH₃ | 1,4-pyrimidin-2-yl | H | (A); 110° C. |
| 189 | B1 | Br | OCH₃ | 1,4-pyrimidin-2-yl | H | (B); 145° C. |
| 195 | B15 | Br | OCH₃ | 3,4-difluorophenyl | H | (A); 250° C. |
| 196 | B15 | Br | OCH₃ | 3,4-difluorophenyl | H | (B); 184° C. |
| 201 | B1 | Br | OCH₃ | 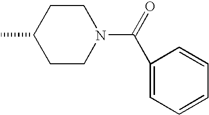 | H | (A); 214° C. |
| 202 | B1 | Br | OCH₃ | 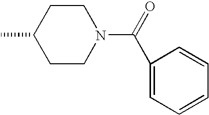 | H | (B); 246° C. |
| 203 | B9 | 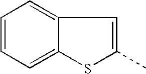 | OCH₃ | 3-fluorophenyl | H | (A); 225° C. |
| 204 | B9 | 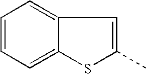 | OCH₃ | 3-fluorophenyl | H | (B); 216° C. |
| 205 | B7 | Br | OCH₃ | 1-naphthyl | F | (A); 213° C. |
| 206 | B7 | Br | OCH₃ | 1-naphthyl | F | (B); 213° C. |
| 207 | B15 | F | OCH₃ | 3,5-difluorophenyl | H | (A); 232° C. |
| 208 | B15 | F | OCH₃ | 3,5-difluorophenyl | H | (B); 188° C. |
| 212 | B7 | 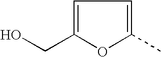 | OCH₃ | 1-naphthyl | H | (B); 220° C. |

TABLE 2

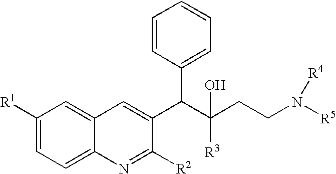

| Comp. nr. | Ex. nr. | R¹ | R² | R³ | R⁴ | R⁵ | Phys. data (salt/melting points) and stereochemistry |
|---|---|---|---|---|---|---|---|
| 18 | B1 | Br | OCH₃ | phenyl | CH₂CH₃ | CH₂CH₃ | •ethanedioate (2:3); (A); 230° C. |
| 19 | B1 | Br | OCH₃ | phenyl | CH₂CH₃ | CH₂CH₃ | •ethanedioate (2:3), (B); 150° C. |
| 44 | B4 | Br | OCH₃ | phenyl | H | H | (A); 190° C. |
| 9 | B4 | Br | OCH₃ | phenyl | H | H | (B); 204° C. |
| 141 | B7 | Br | OCH₃ | 2-naphthyl | CH₃ | CH₂CH₃ | (A); 188° C. |
| 142 | B7 | Br | OCH₃ | 2-naphthyl | CH₃ | CH₂CH₃ | (B); 202° C. |
| 230 | B12 | Br | OCH₃ | 1-naphthyl | CH₃ | benzyl | /oil |
| 147 | B7 | Br | OCH₃ | 1-naphthyl | CH₃ | CH₂CH₃ | (A); 168° C. |
| 148 | B7 | Br | OCH₃ | 1-naphthyl | CH₃ | CH₂CH₃ | (B); 212° C. |
| 56 | B13 | Br | OCH₃ | 1-naphthyl | CH₃ | H | (A); 204° C. |
| 214 | B13 | Br | OCH₃ | 1-naphthyl | CH₃ | H | (B); 225° C. |

TABLE 3

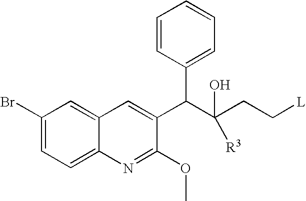

| | | | | Stereo- |
|---|---|---|---|---|
| | | | | chemistry |
| Comp. | Ex. | | | and melting |
| nr. | nr. | R³ | L | points |
| 47 | B1 | phenyl | 1-piperidinyl | (A); 190° C. |
| 48 | B1 | phenyl | 1-piperidinyl | (B); 210° C. |
| 128 | B1 | 2-naphthyl | 1-piperidinyl | (A); 254° C. |
| 129 | B1 | 2-naphthyl | 1-piperidinyl | (B); 212° C. |
| 49 | B1 | phenyl | 1-imidazolyl | (A); 216° C. |
| 50 | B1 | phenyl | 1-imidazolyl | (B); 230° C. |
| 51 | B1 | phenyl | 1-(4-methyl)piperazinyl | (A); 150° C. |
| 52 | B1 | phenyl | 1-(4-methyl)piperazinyl | (B); 230° C. |
| 53 | B1 | phenyl | 1-(1,2,4-triazolyl) | (A); 180° C. |
| 54 | B1 | phenyl | 1-(1,2,4-triazolyl) | (B); 142° C. |
| 55 | B1 | phenyl | thiomorpholinyl | (A); oil |
| 57 | B5 | phenyl | 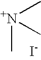 | (A); 244° C. |
| 10 | B5 | phenyl | 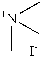 | (B); 198° C. |
| 58 | B6 | phenyl | 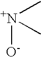 | (A); 208° C. |
| 11 | B6 | phenyl | 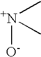 | (B); 208° C. |
| 99 | B11 | 1-naphthyl | 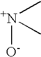 | (A1); 218° C. |
| 100 | B6 | 1-naphthyl | 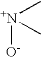 | (A2); 218° C. |
| 101 | B6 | 1-naphthyl | 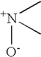 | (B); 175° C. |
| 102 | B5 | 1-naphthyl | 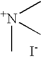 | (A2); 210° C. |
| 103 | B5 | 1-naphthyl | 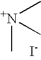 | (B); >250° C. |
| 121 | B5 | 1-naphthyl | 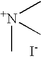 | (A1); 210° C. |
| 123 | B1 | phenyl | morpholinyl | (A); 226° C. |
| 124 | B1 | phenyl | morpholinyl | (B); 210° C. |
| 136 | B7 | 2-naphthyl | 4-methylpyrazinyl | (A); 188° C. |
| 137 | B7 | 2-naphthyl | 4-methylpyrazinyl | (B); 232° C. |
| 139 | B7 | 2-naphthyl | morpholinyl | (A); 258° C. |
| 140 | B7 | 2-naphthyl | morpholinyl | (B); 214° C. |
| 144 | B7 | 2-naphthyl | pyrrolidinyl | (A); 238° C. |
| 145 | B7 | 1-naphthyl | 1-piperidinyl | (A); 212° C. |
| 146 | B7 | 1-naphthyl | 1-piperidinyl | (B); 220° C. |
| 149 | B7 | 1-naphthyl | 4-methylpyrazinyl | (B); 232° C. |
| 151 | B7 | 3-bro- | 4-methylpiperazinyl | (A); 178° C. |
| | | mo-1-naph- | | |
| | | thyl | | |
| 152 | B7 | 3-bro- | 4-methylpiperazinyl | (B); 226° C. |
| | | mo-1-naph- | | |
| | | thyl | | |
| 153 | B7 | 6-bro- | 4-methylpiperazinyl | (A); 208° C. |
| | | mo-2-naph- | | |
| | | thyl | | |
| 154 | B7 | 6-bro- | 4-methylpiperazinyl | (B); 254° C. |
| | | mo-2-naph- | | |
| | | thyl | | |
| 155 | B7 | 6-bro- | 1-piperidinyl | (A); 224° C. |
| | | mo-2-naph- | | |
| | | thyl | | |
| 156 | B7 | 1-naphthyl | 4-methylpiperazinyl | (A); 200° C. |
| 157 | B7 | 6-bro- | 1-pyrrolidinyl | (B); 220° C. |
| | | mo-2-naph- | | |
| | | thyl | | |
| 158 | B7 | 1-naphthyl | morpholinyl | (B); 272° C. |
| 166 | B7 | 6-bro- | 1-piperidinyl | (B); 218° C. |
| | | mo-2-naph- | | |
| | | thyl | | |
| 170 | B7 | 2-naphthyl | 1-pyrrolidinyl | (A); 238° C. |
| 171 | B7 | 2-naphthyl | 1-pyrrolidinyl | (B); 218° C. |
| 172 | B7 | 1-naphthyl | 1,2,4-triazol-1-yl | /142° C. |
| 173 | B7 | 1-naphthyl | 1,2-imidazol-1-yl | (A); 222° C. |
| 177 | B7 | 6-bro- | morpholinyl | (A); 242° C. |
| | | mo-2-naph- | | |
| | | thyl | | |
| 178 | B7 | 6-bro- | morpholinyl | (B); 246° C. |
| | | mo-2-naph- | | |
| | | thyl | | |
| 187 | B7 | 1-naphthyl | 1,2-imidazol-1-yl | (B); 236° C. |
| 200 | B7 | 2-naphthyl | 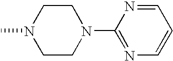 | (A); 254° C. |
| 209 | B7 | 2-naphthyl | 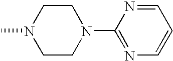 | (B); 198° C. |

TABLE 4

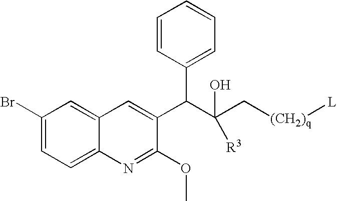

| | | | | | Stereochemistry |
|---|---|---|---|---|---|
| | | | | | and melting |
| Comp. nr. | Ex. nr. | R³ | Q | L | points |
| 61 | B1 | phenyl | 0 | N(CH₃)₂ | (A); 220° C. |
| 62 | B1 | phenyl | 0 | N(CH₃)₂ | (B); 194° C. |
| 63 | B1 | phenyl | 2 | N(CH₃)₂ | (A); 150° C. |
| 64 | B1 | phenyl | 2 | N(CH₃)₂ | (B); 220° C. |
| 125 | B7 | 2-naphthyl | 2 | N(CH₃)₂ | (A); 229° C. |
| 126 | B7 | 2-naphthyl | 2 | N(CH₃)₂ | (B); 214° C. |
| 65 | B1 | phenyl | 3 | N(CH₃)₂ | (A); 130° C. |
| 66 | B1 | phenyl | 3 | N(CH₃)₂ | (B); 170° C. |
| 132 | B7 | 2-naphthyl | 2 | pyrrolidinyl | (A); 227° C. |
| 133 | B7 | 2-naphthyl | 2 | pyrrolidinyl | (B); 222° C. |
| 161 | B7 | 2-naphthyl | 2 | morpholinyl | (B); 234° C. |
| 186 | B7 | 1-naphthyl | 2 | N(CH₃)₂ | (A); 187° C. |
| 190 | B7 | 2-naphthyl | 3 | N(CH₃)₂ | (A); 170° C. |
| 191 | B7 | 2-naphthyl | 3 | N(CH₃)₂ | (B); 145° C. |
| 192 | B7 | 2-naphthyl | 2 | N(CH₂CH₃)₂ | (A); 90° C. |
| 193 | B7 | 2-naphthyl | 2 | N(CH₂CH₃)₂ | (B); 202° C. |
| 194 | B7 | 1-naphthyl | 2 | pyrrolidinyl | (B); 206° C. |
| 197 | B7 | 1-naphthyl | 3 | N(CH₃)₂ | (A); 160° C. |
| 198 | B7 | 2-naphthyl | 2 | morpholinyl | (A); 215° C. |
| 199 | B7 | 1-naphthyl | 2 | N(CH₂CH₃)₂ | (A); 185° C. |
| 210 | B7 | 1-naphthyl | 2 | morpholinyl | (B); 222° C. |
| 211 | B7 | 1-naphthyl | 2 | morpholinyl | (A); 184° C. |

TABLE 5

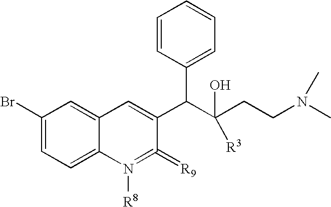

| | | | | | Stereochemistry |
|---|---|---|---|---|---|
| | | | | | and melting |
| Comp. nr. | Ex. nr. | R³ | R⁸ | R⁹ | points |
| 104 | B1 | phenyl | —CH═CH—N═ | | (A); 170° C. |
| 105 | B1 | phenyl | —CH═CH—N═ | | (B); 150° C. |
| 106 | B1 | phenyl | CH₃ | ═O | (A); 224° C. |
| 107 | B1 | phenyl | CH₃ | ═O | (B); 180° C. |
| 138 | B7 | 1-naphthyl | H | ═O | (A1); >260° C. |

TABLE 6

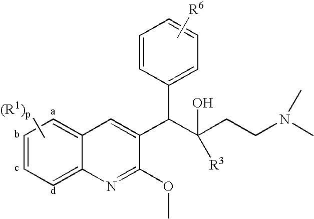

| | | R¹ | | | | | | |
|---|---|---|---|---|---|---|---|---|
| Comp. nr. | Ex. nr. | a | b | c | d | R³ | R⁶ | Stereochemistry and melting points |
| 215 | B9 | H | Br | CH₃ | H | 3-fluorophenyl | H | (A); 197° C. |
| 216 | B9 | H | Br | CH₃ | H | 3-fluorophenyl | H | (B); 158° C. |
| 217 | B7 | H | H | Br | H | 1-naphthyl | H | (A); 212° C. |
| 218 | B7 | H | H | Br | H | 1-naphthyl | H | (B); 172° C. |
| 219 | B9 | H | Br | H | CH₃ | 3-fluorophenyl | H | (A); 220° C. |
| 220 | B9 | H | Br | H | CH₃ | 3-fluorophenyl | H | (B); 179° C. |
| 221 | B7 | Br | H | H | H | 1-naphthyl | H | (A); 170° C. |
| 224 | B7 | Br | H | H | H | 1-naphthyl | H | /205° C. |
| 222 | B7 | H | Br | H | H | 1-naphthyl | 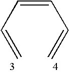 | (A); 155° C. |
| 223 | B7 | H | Br | H | H | 1-naphthyl | 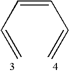 | (B); 205° C. |
| 225 | B7 | H | Br | CH₃ | H | 1-naphthyl | H | (A); 238° C. |
| 226 | B7 | H | Br | CH₃ | H | 1-naphthyl | H | (B); 208° C. |
| 227 | B15 | H | Br | CH₃ | H | 3,5-difluorophenyl | H | (A); 195° C. |
| 228 | B15 | H | Br | CH₃ | H | 3,5-difluorophenyl | H | (B); 218° C. |
| 229 | B7 | H | CH₃ | CH₃ | H | 1-naphthyl | H | (A); 238° C. |

### PHARMACOLOGICAL EXAMPLES

### In-vitro Method for Testing Compounds Against Resistant Mycobacteria Strains

The in vitro activity has been assessed by the determination of the minimal inhibitory concentration (MIC: MIC will be the lowest drug concentration inhibiting more than 99% of the bacterial growth on control medium without antibiotic) in solid medium.

For the in vitro test, the following medium was used: 10% Oleic acid Albumin Dextrose Catalase (OADC)-enriched 7H11 medium.

As inoculum was used: two appropriate dilutions of 10% OADC-enriched 7H9 broth culture aged of 3 to 14 days depending on the mycobacterial species (final inocula=about 10² and 10⁴ cfu (colony forming units))

The incubations were done at 30° C. or 37° C. for 3 to 42 days depending on the mycobacterial species.

Tables 7 and 8 list the MICs (mg/L) against different clinical isolates of resistant Mycobacterium strains. Tables 9 and 10 list the MICs (mg/L) against different clinical isolates of Mycobacterium strains resistant to fluoroquinolones.

In the Tables rifampin and ofloxacin are also included as reference.

TABLE 7

| Strains | Rifampin | Compound 12 | Compound 109 | Compound 2 |
|---|---|---|---|---|
| M.tuberculosis | 0.5 | 0.06 | 0.12 | 0.25 |
| isoniazid-resistant | | | | |
| low level | | | | |
| M.tuberculosis | 0.5 | ≦0.01 | 0.03 | ≦0.01 |
| isoniazid-resistant | | | | |
| high level | | | | |
| M.tuberculosis | >256 | 0.06 | 0.12 | 0.06 |
| rifampin-resistant | | | | |

TABLE 8

| Strains | Rifampin | Compound 12 |
|---|---|---|
| M.tuberculosis | 0.25 | 0.01 |
| isoniazid-resistant | | |
| High Level | | |
| M.tuberculosis | 0.5 | 0.06 |
| isoniazid-resistant | | |
| high level | | |
| M.tuberculosis | 0.12 | 0.03 |
| isoniazid-resistant | | |
| high level | | |
| M.tuberculosis | ≦0.06 | 0.01 |
| isoniazid-resistant | | |
| high level | | |
| M.tuberculosis | 0.25 | 0.01 |
| isoniazid-Resistant | | |
| high level and | | |
| streptomycin- | | |
| resistant | | |
| M.tuberculosis | 256 | 0.03 |
| rifampin-resistant | | |
| M.tuberculosis | 16 | 0.03 |
| rifampin-resistant | | |
| M.tuberculosis | 256 | 0.01 |
| rifampin-resistant | | |
| M.tuberculosis | 0.5 | 0.01 |
| streptomycin- | | |
| resistant | | |
| M.tuberculosis | 0.25 | 0.01 |
| ethambutol- | | |
| resistant | | |
| M.tuberculosis | 0.5 | 0.03 |
| pyrazinamide- | | |
| resistant | | |

TABLE 9

| Strains | Rifampin | Compound 12 | Ofloxacin |
|---|---|---|---|
| M.tuberculosis | 1 | 0.06 | 8 (Ala83Val |
| | | | Ser84Pro)* |
| M.tuberculosis | 2 | 0.12 | 32 (Asp87Gly)* |
| M.avium | 16 | 0.007 | 128 (Ala83Val)* |

*The indications between parentheses indicate the mutations in the protein responsible for ofloxacin resistance

TABLE 10

| Strains | Rifampin | Compound 12 | Ofloxacin |
|---|---|---|---|
| M.smegmatis | 64 | 0.01 | 8 (Asp87Gly)* |
| M.smegmatis | 64 | 0.01 | 32 (Ala83Val |
| | | | and Asp87Gly)* |
| M.smegmatis | 64 | 0.01 | 32 (Ala83Val |
| | | | and Asp87Gly)* |
| M.smegmatis | 128 | 0.007 | 2 (Ala83Val)* |
| M.smegmatis | ND | 0.003 | 32 (Asp87Gly)* |
| M.fortuitum | 128 | 0.01 | 1 |
| M.fortuitum | 128 | 0.007 | 1 (Ser84Pro)* |
| M.fortuitum | >64 | 0.01 | 1.5 (Asp87Gly)* |

*The indications between parentheses indicate the mutations in the protein responsible for ofloxacin resistance.

From these results it can be concluded that the present compounds are highly active against drug resistant Mycobacterium strains. There is no evidence of cross-resistance with antituberculosis drugs: isoniazid, rifampin, streptomycin, ethambutol and pyrazinamide. In the same manner, there is no evidence of cross-resistance with fluoroquinolones.

Compound 12 was also tested against 2 multi-drug resistant M. tuberculosis strains, i.e. a strain resistant to isoniazid 10 mg/L and rifampin and a strain resistant to isoniazid 0.2 mg/L and rifampin. The MIC obtained for compound 12 for both strains is 0.03 mg/L.

### In Vivo Method for Testing Combinations Against M. tuberculosis Infected Mice

Four weeks old Swiss female mice were infected intravenously with 5×10⁶ CFU of M.tuberculosis H37Rv strain. On D1 and D14 following the infection, ten mice were sacrificed to determine the baseline values of spleen weight and CFU counts in the spleens and the lungs after inoculation and at the beginning of treatment. The remaining mice were allocated to the following treatment groups: an untreated control group for survival monitoring, two positive control groups, one with a regimen for susceptible tuberculosis treated with 2 months of isoniazid 25 mg/kg, rifampin 10 mg/kg, pyrazinamide 150 mg/kg daily, and the other with a regimen for multi drug resistant tuberculosis treated with 2 months of daily amikacin 150 mg/kg, ethionamide 50 mg/kg, moxifloxacin 100 mg/kg and pyrazinamide 150 mg/kg. Three negative control groups were treated for 2 months with one of the following drugs, rifampin 10 mg/kg daily, moxifloxacin 100 mg/kg daily and compound 12 25 mg/kg daily. All the tested regimens either for susceptible tuberculosis or for MDR tuberculosis are summarized in table 11. All the groups contained ten mice and were treated during 8 weeks from D14 to D70 five days a week. The parameters used for assessing the severity of infection and the effectiveness of treatments were: survival rate, spleen weight, gross lung lesions and CFU counts in the spleens and in the lungs.

Survival rate : The untreated mice began to die by day 21 after infection and all the mice were dead by day 28 of infection. All the treatments were able to prevent the mortality of mice and few mice died because of accident of gavage.

TABLE 11
Experimental design

| Dates of | | | | | Total |
|---|---|---|---|---|---|
| sacrifices | D-13 | D0 | 1 month | 2 month | mice |
| Controls | | | | | |
| Untreated | 10 | 10 | 10 | | 30 |
| 2 Rifampicin | | | 10 | 10 | 20 |
| 2 Moxifloxacin | | | 10 | 10 | 20 |
| 2 compound 12 | | | 20* | 10 | 30 |
| Positive Controls | | | | | |
| 2 RMP + INH + PZA | | | 10 | 10 | 20 |
| 2 AMIK + ETHIO + MXFX + PZA | | | 10 | 10 | 20 |
| Tested regimens | | | | | |
| (Susceptible TB regimen) | | | | | |
| 2 RMP + INH | | | 10 | 10 | 20 |
| 2 RMP + compound 12 | | | 10 | 10 | 20 |
| 2 INH + compound 12 | | | 10 | 10 | 20 |
| 2 RMP + INH + compound 12 | | | 10 | 10 | 20 |
| 2 INH + PZA + compound 12 | | | 10 | 10 | 20 |
| 2 RMP + PZA + compound 12 | | | 10 | 10 | 20 |
| 2 RMP + INH + PZA + compound 12 | | | 10 | 10 | 20 |
| Tested regimens | | | | | |
| (Resistant TB regimen) | | | | | |
| 2 AMIK + ETHIO + PZA | | | 10 | 10 | 20 |
| 2 AMIK + ETHIO + PZA + compound 12 | | | 10 | 10 | 20 |
| 2 AMIK + MXFX + PZA | | | 10 | 10 | 20 |
| 2 AMIK + MXFX + PZA + compound 12 | | | 10 | 10 | 20 |
| 2 AMIK + ETHIO + MXFX + PZA + compound 12 | | | 10 | 10 | 20 |
| Total | 10 | 10 | 190 | 170 | 380 |

Dosages: Rifampicin (RMP) = 10 mg/kg, Isoniazid (INH) = 25 mg/kg, Pyrazinamide (PZA) = 150 mg/kg, Amikacin (AMIK) = 150 mg/kg, Ethionamide (ETHIO) = 50 mg/kg, Moxifloxacin (MXFX) = 100 mg/kg, compound 12 = 25 mg/kg
* for serum dosage

The following Table shows the results of the 2 month experiment.

TABLE 12
Mean spleen weight and number of CFU per spleen and luna of
M.tuberculosis-infected mice and treated with various
treatments for 2 months.

| | No. | Spleen | Mean CFU (log₁₀) per | |
|---|---|---|---|---|
| Group^{a} | mice | weight (mg) | Spleen | Lung |
| Pretreatment | 10 | 631 ± 121 | 6.40 ± 0.30 | 6.94 ± 0.51 |
| R 10 mg/kg | 9 | 391 ± 70 | 2.75 ± 0.34 | 1.89 ± 0.50 |
| M 100 mg/kg | 10 | 400 ± 99 | 3.53 ± 0.34 | 2.89 ± 0.57 |
| J 25 mg/kg | 8 | 248 ± 47 | 1.24 ± 0.50 | 0.22 ± 0.32 |
| RHZ | 10 | 326 ± 78 | 1.91 ± 0.52 | 0.97 ± 0.61 |
| AEtZM | 10 | 331 ± 86 | 1.60 ± 0.38 | 0.10 ± 0.09 |
| RH | 10 | 400 ± 100 | 2.49 ± 0.42 | 1.09 ± 0.36 |
| RJ | 9 | 304 ± 61 | 2.06 ± 0.61 | 1.63 ± 0.77 |
| HJ | 8 | 293 ± 56 | 1.27 ± 0.31 | 0.36 ± 0.40 |
| RHJ | 9 | 297 ± 74 | 0.64 ± 0.63 | 0.19 ± 0.36 |
| HZJ | 7 | 257 ± 40 | 0.07 ± 0 | 0.07 ± 0 |
| RZJ | 9 | 281 ± 56 | 0.07 ± 0 | 0.07 ± 0 |
| RHZJ | 10 | 265 ± 47 | 0.12 ± 0.15 | 0.07 ± 0 |
| AEtZ | 10 | 344 ± 46 | 2.75 ± 0.25 | 1.20 ± 0.26 |
| AEtZJ | 9 | 331 ± 86 | 0.10 ± 0.10 | 0.07 ± 0 |
| AMZ | 10 | 287 ± 31 | 1.89 ± 0.51 | 0.75 ± 0.55 |
| AMZJ | 8 | 296 ± 63 | 0.07 ± 0 | 0.07 ± 0 |
| AEtMZJ | 8 | 285 ± 53 | 0.07 ± 0 | 0.07 ± 0 |

^{a}Except the pretreatment values were obtained from mice sacrificed on day 14 after inoculation, the remaining results were obtained from mice sacrificed on day 42 after inoculation. Treatment began on day 14, and was administered five time weekly for four weeks.
Isoniazid (H), rifampin (R), moxifloxacin (M), pyrazinamide (Z), compound 12 (J), amikacin (A), ethionamide (Et).

### In Vitro Testing of Susceptibility to Compound 12 of Fully Susceptible and Multi Drug Resistant M. tuberculosis Strains in Solid Medium Assay

The susceptibility to compound 12 of 73 M. tuberculosis strains was tested in a solid medium assay (agar plates). The panel of strains included strains (41) fully susceptible to standard anti-tuberculosis drugs as well as multi drug resistant (MDR) strains (32). i.e. strains resistant to at least rifampin and isoniazid.

Agar plates were welded with solutions containing compound 12 in a concentration ranging from 0.002 mg/L to 0.256 mg/L (8 different concentrations tested). M. tuberculosis isolates were then plated on each agar plate and the plates were sealed and to incubated at 36° C. for 3 weeks.

Isolate growth was analyzed 3 weeks following plate inoculation and an isolate's MIC was defined as the first concentration at which no growth was observed.

For all the tested strains, no growth was seen at concentrations higher than 0.064 mg/L, the majority of strains showed an MIC of 0.032 mg/L.

No difference in MIC was seen between fully susceptible and MDR M. tuberculosis strains.

### In Vivo Testing of Susceptibility of M tuberculosis to Compound 12 in Combination With Other Antimycobacterial Agents

Swiss mice were inoculated intravenously with 10⁶ log colony forming units (CFU) of strain H37Rv. Compound 12 (J) was administrated by gavage 5 days/week (once a day treatment group) or once a week from day 14 to day 70 after inoculation, in monotherapy or in association with isoniazid (H), rifampin (R), pyrazinamide (Z), or moxifloxacin (M). The lung CFU was determined after 1 or 2 months of treatment. The results are gathered in Tables 13 and 14.

TABLE 13
Results for once-a-day group after 1 and 2 months

| | | | % positive | Decrease | Decrease |
|---|---|---|---|---|---|
| | CFU | | mice | 1 mo | 2 mo |
| | 1 month | 2 months | 2nd month | vs D0 | vs D0 |
| D0 | 7.23 | | | | |
| R | 6.01 | 4.07 | 10/10 | −1.22 | −3.16 |
| H | 4.89 | 4.72 | 10/10 | −2.34 | −2.51 |
| Z | 6.17 | 6.43 | 7/7 | −1.06 | −0.8 |
| M | 5.51 | 4.3 | 10/10 | −1.72 | −2.93 |
| J | 4.14 | 2.28 | 8/10 | −3.09 | −4.95 |
| RH | 5.07 | 3.12 | 10/10 | −2.16 | −4.11 |
| RZ | 5.38 | 1.91 | 8/10 | −1.85 | −5.32 |
| HZ | 5.47 | 3.93 | 10/10 | −1.76 | −3.3 |
| RM | 5.52 | 3.13 | 8/10 | −1.71 | −4.1 |
| JR | 4.67 | 1.89 | 7/10 | −2.56 | −5.34 |
| JH | 3.75 | 1.91 | 8/10 | −3.48 | −5.32 |
| JZ | 1.61 | 0 | 0/10 | −5.62 | −7.23 |
| JM | 4.61 | 2.13 | 7/9 | −2.62 | −5.1 |
| RHZ | 3.87 | 2.22 | 9/9 | −3.36 | −5.01 |
| RMZ | 4.59 | 1.36 | 8/10 | −2.64 | −5.87 |
| JHZ | 1.71 | 0.18 | 2/9 | −5.52 | −7.05 |
| JHR | 4.37 | 1.15 | 8/10 | −2.86 | −6.08 |
| JMR | 4.42 | 1.37 | 8/9 | −2.81 | −5.86 |
| JRZ | 2.31 | 0.07 | 3/10 | −4.92 | −7.16 |
| JMZ | 1.44 | 0.03 | 2/9 | −5.79 | −7.2 |

TABLE 14
Results for once-a-week group after 2 months

| | lung CFU* | % positive mice |
|---|---|---|
| DO | 7.23 | |
| J | 1.99 ± 0.75 | 9/9 |
| M | 6.44 ± 0.5 | 7/7 |
| P | 3.26 ± 0.58 | 10/10 |
| JP | 1.63 ± 0.92 | 8/9 |
| JPM | 1.85 ± 0.7 | 10/10 |
| JPH | 11.48 ± 0.79 | 10/10 |
| JPZ | 0.23 ± 0.72 | 1/10 |

1. Use of a substituted quinoline derivative for the preparation of a medicament for the treatment of an infection with a drug resistant Mycobacterium strain wherein the substituted quinoline derivative is a compound according to Formula (Ia) or Formula (Ib) 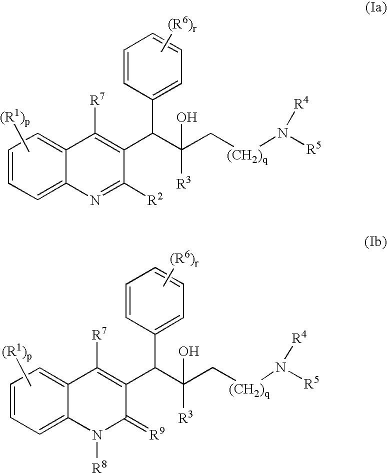
a pharmaceutically acceptable acid or base addition salt thereof, a stereochemically isomeric form thereof, a tautomeric form thereof or a N-oxide form thereof, wherein:
R¹ is hydrogen, halo, haloalkyl, cyano, hydroxy, Ar, Het, alkyl, alkyloxy, alkylthio, alkyloxyalkyl, alkylthioalkyl, Ar-alkyl or di(Ar)alkyl;
p is an integer equal to 1, 2, 3 or 4;
R² is hydrogen, hydroxy, mercapto, alkyloxy, alkyloxyalkyloxy, alkylthio, mono or di(alkyl)amino or a radical of formula 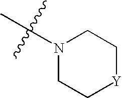
wherein Y is CH₂, O, S, NH or N-alkyl;
R³ is alkyl, Ar, Ar-alkyl Het or Het-alkyl;
q is an integer equal to zero, 1, 2, 3 or 4;
R⁴ and R⁵ each independently are hydrogen, alkyl or benzyl; or
R⁴ and R⁵ together and including the N to which they are attached may form a radical selected from the group of pyrrolidinyl, 2H-pyrrolyl 2-pyrrolinyl, 3-pyrrolinyl, pyrrolyl, imidazolidinyl, pyrazolidinyl, 2-imidazolinyl, 2-pyrazolinyl, imidazolyl, pyrazolyl, triazolyl, piperidinyl, pyridinyl, piperazinyl imidazolidinyl, pyridazinyl, pyrimidinyl, pyrazinyl triazinyl, morpholinyl and thiomorpholinyl, optionally substituted with alkyl, halo, haloalkyl, hydroxy, alkyloxy, amino, mono- or dialkylamino, alkylthio, alkyloxyalkyl, alkylthioalkyl and pyrimidinyl;
R⁶ is hydrogen, halo, haloalkyl, hydroxy, Ar, alkyl, alkyloxy, alkylthio, alkyloxyalkyl alkylthioalkyl, Ar-alkyl or di(Ar)alkyl; or
two vicinal R⁶ radicals may be taken together to form a bivalent radical of formula —CH═CH—CH═CH—;
r is an integer equal to 1, 2, 3, 4 or 5; and
R⁷ is hydrogen, alkyl, Ar or Het;
R⁸ is hydrogen or alkyl;
R⁹ is oxo; or
R⁸ and R⁹ together form the radical ═N—CH═CH—;
alkyl is a straight or branched saturated hydrocarbon radical having from 1 to 6 carbon atoms; or is a cyclic saturated hydrocarbon radical having from 3 to 6 carbon atoms; or is a a cyclic saturated hydrocarbon radical having from 3 to 6 carbon atoms attached to a straight or branched saturated hydrocarbon radical having from 1 to 6 carbon atoms; wherein each carbon atom can be optionally substituted with halo, hydroxy, alkyloxy or oxo;
Ar is a homocycle selected from the group of phenyl, naphthyl, acenaphthyl, tetrahydronaphthyl, each optionally substituted with 1, 2 or 3 substituents, each substituent independently selected from the group of hydroxy, halo, cyano, nitro, amino, mono- or dialkylamino, alkyl, haloalkyl, alkyloxy, haloalkyloxy, carboxyl, alkyloxycarbonyl, aminocarbonyl, morpholinyl and mono- or dialkylaminocarbonyl;
Het is a monocyclic heterocycle selected from the group of N-phenoxypiperidinyl, pyrrolyl, pyrazolyl, imidazolyl, furanyl, thienyl, oxazolyl, isoxazolyl, thiazolyl, isothiazolyl, pyridinyl, pyrimidinyl, pyrazinyl and pyridazinyl; or a bicyclic heterocycle selected from the group of quinolinyl, quinoxalinyl, indolyl, benzimidazolyl, benzoxazolyl, benzisoxazolyl, benzothiazolyl, benzisothiazolyl, benzofuranyl, benzothienyl, 2,3-dihydrobenzo[1,4]dioxinyl or benzo[1,3]dioxolyl; each monocyclic and bicyclic heterocycle may optionally be substituted on a carbon atom with 1, 2 or 3 substituents selected from the group of halo, hydroxy, alkyl or alkyloxy;
halo is a substituent selected from the group of fluoro, chloro, bromo and iodo and
haloalkyl is a straight or branched saturated hydrocarbon radical having from 1 to 6 carbon atoms or a cyclic saturated hydrocarbon radical having from 3 to 6 carbon atoms, wherein one or more carbon atoms are substituted with one or more halo-atoms.

2. Use according to claim 1 wherein R⁶ in Formula (Ia) or (Ib) is hydrogen, halo, haloalkyl, hydroxy, Ar, alkyl, alkyloxy, alkylthio, alkyloxyalkyl, alkylthioalkyl, Ar-alkyl or di(Ar)alkyl.

3. Use according to claim 1 or 2 wherein in Formula (Ia) or (Ib) R¹ is halo.

4. Use according to any one of the preceding claims wherein in Formula (Ia) or (Ib) p is equal to 1.

5. Use according to any one of the preceding claims wherein in Formula (Ia) or (Ib) R² is alkyloxy.

6. Use according to any one of the preceding claims wherein in Formula (Ia) or (Ib) R³ is naphthyl or phenyl, each optionally substituted with halo.

7. Use according to claim 6 wherein R³ is naphthyl.

8. Use according to any one of the preceding claims wherein in Formula (Ia) or (Ib) q is equal to 1.

9. Use according to any one of the preceding claims wherein in Formula (Ia) or (Ib) R⁴ and R⁵ each independently are hydrogen or alkyl.

10. Use according to claim 9 wherein R⁴ and R⁵ each independently are C₁₋₄alkyl.

11. Use according to any one of the preceding claims wherein in Formula (Ia) or (Ib) R⁶ is hydrogen.

12. Use according to any one of the preceding claims wherein in Formula (Ia) or (Ib) R⁷ is hydrogen.

13. Use according to claim 1, characterized in that the compound is selected from the group consisting of:
1-(6-bromo-2-methoxy-quinolin-3-yl)-2-(3,5-difluoro-phenyl)-4-dimethylamino-1-phenyl-butan-2-ol;
1-(6-bromo-2-methoxy-quinolin-3-yl)-4-dimethylamino-2-naphthalen-1-yl-1-phenyl-butan-2-ol;
1-(6-bromo-2-methoxy-quinolin-3-yl)-2-(2,5-difluoro-phenyl)-4-dimethylamino-1-phenyl-butan-2-ol;
1-(6-bromo-2-methoxy-quinolin-3-yl)-2-(2,3-difluoro-phenyl)-4-dimethylamino-1-phenyl-butan-2-ol;
1-(6-bromo-2-methoxy-quinolin-3-yl)-4-dimethylamino-2-(2-fluoro-phenyl)-1-phenyl-butan-2-ol;
1-(6-bromo-2-methoxy-quinolin-3-yl)-4-dimethylamino-2-naphthalen-1-yl-1-p-tolyl-butan-2-ol;
1-(6-bromo-2-methoxy-quinolin-3-yl)-4-methylamino-2-naphthalen-1-yl-1-phenyl-butan-2-ol;
1-(6-bromo-2-methoxy-quinolin-3-yl)-4-dimethylamino-2-(3-fluoro-phenyl)-1-phenyl-butan-2-ol; and
1-(6-bromo-2-methoxy-quinolin-3-yl)-4-dimethylamino-2-phenyl-1-phenyl-butan-2-ol;
a pharmaceutically acceptable acid or base addition salt thereof, a stereochemically isomeric form thereof, a tautomeric form thereof or a N-oxide form thereof.

14. Use according to claim 13 wherein the compound is selected from the group consisting of
1-(6-bromo-2-methoxy-quinolin-3-yl)-4-dimethylamino-2-(3-fluoro-phenyl)-1-phenyl-butan-2-ol;
1-(6-bromo-2-methoxy-quinolin-3-yl)-4-dimethylamino-2-phenyl-1-phenyl-butan-2-ol;
1-(6-bromo-2-methoxy-quinolin-3-yl)-4-dimethylamino-2-naphthalen-1-yl-1-phenyl-butan-2-ol;
a pharmaceutically acceptable acid or base addition salt thereof, a stereochemically isomeric form thereof, a tautomeric form thereof or a N-oxide form thereof.

15. Use according to claim 1 wherein the compound is 6-bromo-α-[2-(dimethylamino)ethyl]-2-methoxy-α-1-naphthalenyl-β-phenyl-3-quinolineethanol, a pharmaceutically acceptable acid or base addition salt thereof, a stereochemically isomeric forms thereof, a tautomeric form thereof or a N-oxide form thereof.

16. Use according to claim 15 wherein the compound is
6-bromo-α-[2-(dimethylamino)ethyl]-2-methoxy-α-1-naphthalenyl-β-phenyl-3-quinolineethanol, or a pharmaceutically acceptable acid addition salt thereof.

17. Use according to claim 15 wherein the compound is
6-bromo-α-[2-(dimethylamino)ethyl]-2-methoxy-α-1-naphthalenyl-β-phenyl-3-quinolineethanol, or a stereochemically isomeric form thereof.

18. Use according to claim 15 wherein the compound is
6-bromo-α-[2-(dimethylamino)ethyl]-2-methoxy-α-1-naphthalenyl-β-phenyl-3-quinolineethanol, or a N-oxide form thereof.

19. Use according to claim 15 wherein the compound is
(αS, βR)-6-bromo-α-[2-(dimethylamino)ethyl]-2-methoxy-α-1-naphthalenyl-β-phenyl-3-quinolineethanol, or a pharmaceutically acceptable acid addition salt thereof.

20. Use according to claim 19 wherein the compound is
(αS, βR)-6-bromo-α-[2-(dimethylamino)ethyl]-2-methoxy-α-1-naphthalenyl-β-phenyl-3-quinolineethanol.

21. Use according to any one of the preceding claim wherein the drug resistant Mycobacterium strain is multi drug resistant.

22. Use according to any one of the preceding claims wherein the Mycobacterium strain is a Mycobacterium tuberculosis strain.

23. A combination of (a) a compound of formula (Ia) or (Ib) as defined in any one of claims 1 to 20 and (b) one or more other antimycobacterial agents.

24. A combination of (a) a compound of formula (Ia) or (Ib) as defined in any one of claims 1 to 20 and (b) one or more other antimycobacterial agents for use as a medicine.

25. A pharmaceutical composition comprising a pharmaceutically acceptable carrier and, as active ingredient, a therapeutically effective amount of (a) a compound of formula (Ia) or (Ib) as defined in any one of claims 1 to 20 and (b) one or more other antimycobacterial agents.

26. A product containing (a) a compound of formula (Ia) or (Ib) as defined in any one of claims 1 to 20, and (b) one or more other antimycobacterial agents, as a combined preparation for simultaneous, separate or sequential use in the treatment of mycobacterial diseases.

27. A combination, a pharmaceutical composition or a product as claimed in any one of claims 23 to 26 wherein the one or more other antimycobacterial agents comprise pyrazinamide.

28. A combination, a pharmaceutical composition or a product as claimed in any one of claims 23 to 27 wherein the compound of formula (Ia) or (Ib) is (αS, βR)-6-bromo-α-[2-(dimethylamino)ethyl]-2-methoxy-α-1-naphthalenyl-β-phenyl-3-quinolineethanol or a pharmaceutically acceptable acid addition salt thereof.

29. A combination, a pharmaceutical composition or a product as claimed in any one of claims 23 to 28 wherein the compound of formula (Ia) or (Ib) is (αS, βR)-6-bromo-α-[2-(dimethylamino)ethyl]-2-methoxy-α-1-naphthalenyl-β-phenyl-3-quinolineethanol.

30. Use of a combination, a pharmaceutical composition or a product as claimed in any one of claims 23 to 29 for the treatment of an infection with a drug resistant Mycobacterium strain.

31. Use according to claim 30 wherein the drug resistant Mycobacterium strain is a drug resistant M. tuberculosis strain.
